(19) United States
(12) Patent Application Publication
(10) Pub. No.: US 2022/0400794 A1
(43) Pub. Date: Dec. 22, 2022

(54) GARMENTS, ARTICLES OF CLOTHING, AND BODY EQUIPMENT SYSTEMS HAVING A BLADDER SYSTEM

(71) Applicant: Anthony T. Ngo, Cypress, TX (US)

(72) Inventor: Anthony T. Ngo, Cypress, TX (US)

(73) Assignee: ACE PT Products and Equipment, INC, Cypress, TX (US)

(21) Appl. No.: 17/712,186

(22) Filed: Apr. 4, 2022

Related U.S. Application Data
(63) Continuation-in-part of application No. 17/575,634, filed on Jan. 14, 2022.
(60) Provisional application No. 63/136,767, filed on Jan. 13, 2021.
(60) Provisional application No. 63/136,774, filed on Jan. 13, 2021.

(51) Int. Cl. A41D 27/02; A41D 13/00; A41D 31/04

(52) U.S. Cl. CPC A41D 27/02; A41D 13/0015; A41D 31/04; A41D 2400/38

(57) ABSTRACT

The present invention is garments, articles of clothing, and body equipment systems having a bladder system for providing better fit of garments, articles of clothing, and body equipment systems to certain applicable body part(s) or area(s) of a user during use, and for providing greater support, comfort and/or stability to certain applicable body part(s) or area(s) of a user during use. As a non-limiting example, the present invention is compression garments having a clothing component and a bladder system, compression articles of clothing having a clothing component and a bladder system, and also to compression body equipment systems having a bladder system for providing better fit of compression garments, compression articles of clothing, and compression body equipment systems to certain applicable body part(s) or area(s) of a user during use, and for providing greater support, comfort and/or stability to certain applicable body part(s) or area(s) of a user during use.

### CROSS-REFERENCE TO RELATED APPLICATIONS

The present application is a continuation-in-part application of and claims the priority benefit of U.S. Nonprovisional patent application Ser. No. 17/575,634, filed on Jan. 14, 2022, which claims the priority benefit of both U.S. Provisional Patent Application Ser. No. 63/136,767, filed on Jan. 13, 2021, and also of U.S. Provisional Patent Application Ser. No. 63/136,774, filed on Jan. 13, 2021, which are all incorporated herein by reference in their entireties.

### BACKGROUND OF THE INVENTION

### Field of the Invention

The present invention generally relates to garments and articles of clothing, compression garments and articles of clothing, garments and articles of clothing having bladder systems, compression garments and articles of clothing having bladder systems, body equipment systems, and body equipment systems having bladder systems. More specifically, the present invention is directed to garments, articles of clothing, and body equipment systems having a bladder system for providing better fit of the garments, articles of clothing, and body equipment systems to certain applicable body part(s) or area(s) of a user during use, and for providing greater support, comfort and/or stability to certain applicable body part(s) or area(s) of a user during use. As a non-limiting example, the present invention is directed to compression garments having a clothing component and a bladder system, compression articles of clothing having a clothing component and a bladder system, and also to compression body equipment systems having a bladder system for providing better fit of the compression garments, compression articles of clothing, and compression body equipment systems to certain applicable body part(s) or area(s) of a user during use, and for providing greater support, comfort and/or stability to certain applicable body part(s) or area(s) of a user during use.

### Description of the Related Art

Compression garments and articles of clothing, garments and articles of clothing having bladder systems, and compression garments and articles of clothing having bladder systems are known in the art.

The present invention overcomes one or more of the shortcomings of known compression garments and articles of clothing, garments and articles of clothing having bladder systems, and compression garments and articles of clothing having bladder systems. The Applicant is unaware of inventions or patents, taken either singly or in combination, which are seen to describe the present invention as claimed.

An advantage of the present invention is that it allows for a user to readily adjust different levels of the bladder system for providing better fit of the garment or article of clothing and compression garment or article of clothing to certain applicable body part(s) or area(s) of a user during use.

Another advantage of the present invention is that it allows for a user to readily adjust different levels of the bladder system for providing greater support, comfort and/or stability to certain applicable body part(s) or area(s) of a user during use.

A further advantage of the present invention is that it allows for a user to use only one garment or article of clothing or only one compression garment or article of clothing for providing better fit of the garment or article of clothing or compression garment or article of clothing to certain applicable body part(s) or area(s) of a user during use.

An additional advantage of the present invention is that it allows for a user to use only one garment or article of clothing or only one compression garment or article of clothing for providing greater support, comfort and/or stability to certain applicable body part(s) or area(s) of a user during use.

### SUMMARY OF THE INVENTION

The present invention is directed to garments, articles of clothing, and body equipment systems having a bladder system for providing better fit of the garments, articles of clothing, and body equipment systems to certain applicable body part(s) or area(s) of a user during use, and for providing greater support, comfort and/or stability to certain applicable body part(s) or area(s) of a user during use. As a non-limiting example, the present invention is directed to compression garments having a clothing component and a bladder system, compression articles of clothing having a clothing component and a bladder system, and also to compression body equipment systems having a bladder system for providing better fit of the compression garments, compression articles of clothing, and compression body equipment systems to certain applicable body part(s) or area(s) of a user during use, and for providing greater support, comfort and/or stability to certain applicable body part(s) or area(s) of a user during use.

One aspect of the present invention is a garment or article of clothing having a clothing component and a bladder system for providing better or greater fit, protection, support, comfort and/or stability to certain applicable body part(s) or area(s) of a user or wearer during use.

Another aspect of the present invention is a compression garment or article of clothing having a clothing component and a bladder system for providing better or greater fit, protection, support, comfort and/or stability to certain applicable body part(s) or area(s) of a user or wearer during use.

As a non-limiting example or preferred embodiment of a compression garment or article of clothing, the clothing component may be a compression or non-compression top clothing component (such as, but not limited to, a shirt (sleeveless, short sleeve, or long sleeve), a sweatshirt (sleeveless, short sleeve, or long sleeve), a jogging suit top, a sport's jersey, a sport's sweater, a vest, a jacket, a sweater, or a coat) and/or a compression or non-compression bottom clothing component (such as, but not limited to, a casual short, an athletic or sport short (such as, but not limited to, a bicycling short, a lacrosse short, or a rugby short), a casual pant, an athletic or sport pant (such as, but not limited to, a bicycling pant, a baseball pant, or a football pant), a sweatpant, a jogging suit bottom, a legging, or a sock).

The bladder system may be an air bladder system, a fluid bladder system, a combination air and fluid bladder system, any bladder system known to OOSIA, or any combination thereof.

Preferably and as a non-limiting example, the bladder system is an air bladder system that comprises at least one bladder wherein each bladder has at least one chamber dimensioned and configured for receiving air from an air source and adapted for being increased or decreased according to the user's preference and/or need of compression forces directed to certain applicable body part(s) or area(s) of a user or wearer during use to obtain better or greater fit, protection, support, comfort and/or stability to the applicable body part(s) or area(s) of the user. As a non-limiting example, the at least one bladder has multiple, separated chambers positioned at different locations along the bladder so that different applicable body part(s) or area(s) of the user can possibly receive different amounts of compression forces directed to the applicable body part(s) or area(s) during use. As a non-limiting example, the user may wish to receive a higher amount of compression forces directed to the user's back as compared to the user's sides or front.

Alternatively, the air bladder system comprises multiple bladders wherein each bladder has at least one chamber.

As a non-limiting example, the at least one bladder may be interwoven within and/or between the fabric(s) or material(s) (or between layers of the fabric(s) or material(s)) of the clothing component.

Alternatively, the at least one bladder may be secured or positioned between two or more layers of the fabric or material of the clothing component, or may be secured or positioned in front of or behind the fabric or material of the clothing component.

Another aspect of the present invention is a body equipment system having a body equipment component and a bladder system for providing better or greater fit, protection, support, comfort and/or stability to certain applicable body part(s) or area(s) of a user or wearer during use.

As a non-limiting example or preferred embodiment of a body equipment system, the body equipment component may be a compression or non-compression top equipment component (such as, but not limited to, any helmet (such as, but not limited to, a football helmet, a baseball/softball helmet, a skateboarding helmet, a skiing helmet, a motorcycle helmet, a bicycle helmet, or a construction worker helmet), a headgear, a neck protector, a throat protector, an arm sleeve, a football shoulder pad, or a baseball catcher chest protector) and/or a compression or non-compression bottom equipment component (such as, but not limited to, a baseball catcher leg protector).

The bladder system may be an air bladder system, a fluid bladder system, a combination air and fluid bladder system, any bladder system known to OOSIA, or any combination thereof. Preferably, the bladder system of the body equipment system is substantially similar to or exactly the same as the bladder system of the body equipment system.

### BRIEF DESCRIPTION OF THE DRAWINGS

FIG. 1A illustrates a caregiver aiding a movement of a patient.

FIG. 1B illustrates an implementation of a compression vest system, according to an aspect of the present disclosure.

FIGS. 2A and 2B illustrate front and rear perspective views of a vest of a compression vest system, according to an aspect of the present disclosure.

FIG. 3 illustrates a front elevation view of a vest in a closed state, according to an aspect of the present disclosure.

FIG. 4 illustrates a front elevation view of a vest in a first open state, according to an aspect of the present disclosure.

FIG. 5 is an enlarged view of a portion of FIG. 4, identified as Detail 5.

FIG. 6 is an enlarged view of a portion of FIG. 4, identified as Detail 6.

FIG. 7 illustrates an elevation view of a front of a vest in a second open state, according to an aspect of the present disclosure.

FIG. 8 is an enlarged view of a portion of FIG. 3, identified as Detail 8.

FIG. 9 is an enlarged view of a portion of FIG. 4, identified as Detail 9.

FIG. 10 is an enlarged view of a portion of FIG. 4, identified as Detail 10.

FIG. 11 illustrates an elevation view of a back region of a vest, according to an aspect of the present disclosure.

FIG. 12A illustrates an elevation view of an interior surface of a belt of a compression vest system in a passive state, according to an aspect of the present disclosure.

FIG. 12B illustrates an elevation view of an exterior surface of a belt of a compression system in a passive state, according to an aspect of the present disclosure.

FIG. 12C illustrates an elevation view of an exterior surface of a belt of a compression system in an active state, according to an aspect of the present disclosure.

FIGS. 13A and 13B illustrate front and rear perspective views of a compression vest system, according to an aspect of the present disclosure.

FIG. 13C illustrates a front perspective view of a compression vest system and sagittal, coronal, and transverse body planes.

FIG. 14 illustrates a side elevation view of a vest, according to an aspect of the present disclosure.

FIG. 15A illustrates an elevation view of a side of a vest in a closed state, according to an aspect of the present disclosure.

FIG. 15B illustrates an elevation view of a side of a vest in an open state, according to an aspect of the present disclosure.

FIG. 16 illustrates a side elevation view of a vest in a closed state, according to an aspect of the present disclosure.

FIG. 17 illustrates a compression vest according to an aspect of the present disclosure.

FIG. 18 illustrates a compression belt according to an aspect of the present disclosure.

FIGS. 19A and 19B overhead views of a cloak, according to aspect of the present disclosure.

FIG. 20 illustrates an implementation of an integrated compression vest system, according to aspect of the present disclosure.

FIGS. 21A and 21B illustrate front and rear perspective views of a partial implementation of a compression vest system, according to an aspect of the present disclosure.

FIGS. 22A and 22B illustrate front and rear perspective views of a full implementation of a compression vest system, according to an aspect of the present disclosure.

FIGS. 23A and 23B illustrate elevation views of an exterior and an interior of a belt in a compact state.

FIGS. 23C and 23D illustrate elevation views of an exterior and an interior of a belt in an expanded state.

FIG. 24 illustrates an elevation view of a belt including removable stays, according to an aspect of the present disclosure.

FIG. 25 illustrates a front isometric view of a stay.

FIG. 26 illustrates an enlarged view of a portion of FIG. 4 including a zipper.

FIG. 27 illustrates an enlarged view of a portion of FIG. 4 including a hoop and loop access.

FIG. 28 illustrates an elevation view of a belt including narrow sized stays, according to an aspect of the present disclosure.

FIG. 29 illustrates an elevation view of a belt including intermediate sized stays, according to an aspect of the present disclosure.

FIG. 30 illustrates an elevation view of a belt including short stays, according to an aspect of the present disclosure.

FIG. 31 illustrates an elevation view of a belt including wide stays and an anterior segmented pouch, according to an aspect of the present disclosure.

FIG. 32 illustrates an elevation view of a belt, according to an aspect of the present disclosure.

FIGS. 33A and 33B illustrate a front and rear elevation views of a modular segmented pouch, according to an aspect of the present disclosure.

FIG. 34 illustrates an elevation view of a belt with a modular pouch attached, according to an aspect of the present disclosure.

FIG. 35 illustrates an elevation view of a belt with a modular pouch attached, according to an aspect of the present disclosure.

FIG. 36 illustrates an elevation view of a belt including anterior pocket, according to an aspect of the present disclosure.

FIG. 37 illustrates a modular pouch, according to an aspect of the present disclosure.

FIGS. 38A and 38B illustrate front and rear perspective views of a full implementation of a compression vest system including an extra-wide stay and a modular segmented pouch.

FIG. 39 illustrates an elevation view of a belt including an extended central attachment segment, according to an aspect of the present disclosure.

FIG. 40 illustrates a modular segmented pouch, according to an aspect of the present disclosure.

FIGS. 41A and 41B illustrate front and rear perspective views of a full implementation of a compression vest system including a belt with multiple modular segmented pouches, according to an aspect of the present disclosure.

FIG. 42 illustrates a front view of a compression vest system according to an aspect of the present disclosure, wherein the compression vest system is in a closed position.

FIG. 43 illustrates a front, left side, perspective view of the compression vest system of FIG. 42, wherein the zipper expansion and retraction mechanism of the vest is in a closed position while the zipper expansion and retraction mechanism of the belt is in an open position.

FIG. 44 illustrates a front, left side, perspective view of the compression vest system of FIG. 42, wherein the vest is in a partially open position while the zipper expansion and retraction mechanism of the vest is in a closed position.

FIG. 45 illustrates a right side view of the compression vest system of FIG. 42, wherein the belt is in an open position while the zipper expansion and retraction mechanism of the vest is in an open position.

FIG. 46 illustrates a front view of the compression vest system of FIG. 42, wherein the compression vest system is in an open position.

FIG. 47 illustrates a front view of the vest of the compression vest system of FIG. 42, wherein the vest is in the open position.

FIG. 48 illustrates a front, right side view of the vest of the compression vest system of FIG. 42, wherein the right side of the vest is in the open position.

FIG. 49 illustrates a front, left side view of the vest of the compression vest system of FIG. 42, wherein the left side of the vest is in the open position.

FIG. 50 illustrates a front view of a compression vest system according to an aspect of the present disclosure, wherein the compression vest system is in a closed position.

FIG. 51 illustrates a front, right side, perspective view of the compression vest system of FIG. 50, wherein the zipper expansion and retraction mechanism of the vest is in an open position and the zipper expansion and retraction mechanism of the belt is in an open position.

FIG. 52 illustrates a front, left side, perspective view of the compression vest system of FIG. 50, wherein the zipper expansion and retraction mechanism of the vest is in a closed position and the zipper expansion and retraction mechanism of the belt is in a closed position.

FIG. 53 illustrates a front, right side, perspective view of the belt of the compression vest system of FIG. 50, wherein the zipper expansion and retraction mechanism of the belt is in an open position.

FIG. 54 illustrates a front, right side, perspective view of the vest of the compression vest system of FIG. 50, wherein the zipper expansion and retraction mechanism of the vest is in an open position.

FIG. 55 illustrates a rear view of the compression vest system of FIG. 50.

FIG. 56 illustrates a rear, right side view of the compression vest system of FIG. 50, wherein the zipper expansion and retraction mechanism of the vest is in an open position and the zipper expansion and retraction mechanism of the belt is in an open position.

FIG. 57 illustrates a rear, left side view of the compression vest system of FIG. 50, wherein the zipper expansion and retraction mechanism of the vest is in a closed position and the zipper expansion and retraction mechanism of the belt is in a closed position.

FIG. 58 illustrates a rear, left side view of the vest of the compression vest system of FIG. 50, wherein the zipper expansion and retraction mechanism of the vest is in a closed position.

FIG. 59 illustrates a front view of a compression binding vest system according to an aspect of the present disclosure, wherein the compression binding vest system is in a closed position.

FIG. 60 illustrates a rear view of the compression binding vest system of FIG. 59, wherein the zipper expansion and retraction mechanism of the left side of the upper belt is in a closed position and the zipper expansion and retraction mechanism of the left side of the lower belt is in an open position.

FIG. 61 illustrates a rear view of the compression binding vest system of FIG. 59, wherein the zipper expansion and retraction mechanism of the left side of the upper belt is in the closed position and the zipper expansion and retraction mechanism of the left side of the lower belt is in the open position.

FIG. 62 illustrates a rear view of the compression binding vest system of FIG. 59, wherein the zipper expansion and retraction mechanism of the right side of the upper belt is in the open position and the zipper expansion and retraction mechanism of the right side of the lower belt is in the open position.

FIG. 63 illustrates a front, right side view of an upper belt of the compression binding vest system of FIG. 59.

FIG. 64 illustrates a front, left side view of an upper belt of the compression binding vest system of FIG. 59.

FIG. 65 illustrates a rear, right side view of the compression binding vest system of FIG. 59, wherein the zipper expansion and retraction mechanism of the right side of the upper belt is in the open position.

FIG. 66 illustrates a rear, left side view of the compression binding vest system of FIG. 59, wherein the zipper expansion and retraction mechanism of the left side of the upper belt is in the closed position.

FIG. 67 illustrates a front, environmental view of a compression binding vest system according to an aspect of the present disclosure, wherein the compression binding vest system is in a closed position.

FIG. 68 illustrates a rear, environmental view of the compression binding vest system of FIG. 67, wherein the compression binding vest system is in the closed position.

FIG. 69 illustrates a left side view of the compression binding vest system of FIG. 67, wherein the compression binding vest system is in the closed position.

FIG. 70 illustrates a front, environmental view of a compression binding vest system according to an aspect of the present disclosure, wherein the compression binding vest system is in a closed position.

FIG. 71 illustrates a rear, environmental view of the compression binding vest system of FIG. 70, wherein the compression binding vest system is in the closed position.

FIG. 72 illustrates a left side view of the compression binding vest system of FIG. 70, wherein the compression binding vest system is in the closed position.

FIG. 73 illustrates a right side view of the compression binding vest system of FIG. 70, wherein the compression binding vest system is in the closed position.

FIG. 74 illustrates a front view of a leg attachment device according to an aspect of the present disclosure.

FIG. 75 illustrates an environmental, rear, right side view of the leg attachment device of FIG. 74.

FIG. 76 illustrates an environmental, left side view of the leg attachment device of FIG. 74.

FIG. 77 illustrates an environmental, rear, left side view of the leg attachment device of FIG. 74.

FIG. 78 illustrates a perspective, front, right side view of a vest and bladder system according to an aspect of the present disclosure.

FIG. 79 illustrates a perspective, front, bottom, right side view of the vest and bladder system of FIG. 78.

FIG. 80 illustrates a perspective, front, right side, upright view of the vest and bladder system of FIG. 78.

FIG. 81 illustrates a perspective, front, bottom, right side view of the vest and bladder system of FIG. 78.

FIG. 82 illustrates a perspective, front, bottom view of the vest and bladder system of FIG. 78.

FIG. 83 illustrates a perspective, front, bottom, right side view of another vest and bladder system according to an aspect of the present disclosure.

FIG. 84 illustrates a perspective, front, left side, upright view of the vest and bladder system of FIG. 83.

FIG. 85 illustrates a perspective, front, right side, upright view of the vest and bladder system of FIG. 83.

FIG. 86 illustrates a perspective, front, bottom view of a further vest and bladder system according to an aspect of the present disclosure.

FIG. 87 illustrates a perspective, rear, bottom view of the vest and bladder system of FIG. 86.

FIG. 88 illustrates a left side view of the vest and bladder system of FIG. 86.

FIG. 89 illustrates a perspective, front view of a bladder system according to an aspect of the present disclosure.

FIG. 90 illustrates a rear view of a cervical and head region of a skeleton.

FIG. 91 illustrates a perspective, front view of another vest and bladder system according to an aspect of the present disclosure.

FIG. 92 illustrates a perspective, front view of the vest and bladder system of FIG. 91.

FIG. 93 illustrates a perspective, front view of a further vest and bladder system according to an aspect of the present disclosure.

FIG. 94 illustrates a perspective, front view of the vest and bladder system of FIG. 93.

FIG. 95 illustrates a front view of an upper section of the vest and bladder system of FIG. 93.

FIG. 96 illustrates a front view of a head and neck bladder device of the vest and bladder system of FIG. 93.

FIG. 97 illustrates a front view of the head and neck bladder device of FIG. 96.

FIG. 98 illustrates a front view of the head and neck bladder device of FIG. 96.

FIG. 99 illustrates a front view of the head and neck bladder device of FIG. 96.

FIG. 100 illustrates a front view of the head and neck bladder device of FIG. 96.

FIG. 101 illustrates a front view of the head and neck bladder device of FIG. 96.

FIG. 102 illustrates a front view of the head and neck bladder device of FIG. 96.

FIG. 103 illustrates a front view of the head and neck bladder device of FIG. 96.

FIG. 104 illustrates an environmental, rear view of the vest and bladder system of FIG. 93.

FIG. 105 illustrates an environmental, perspective, rear, upper right side view of the vest and bladder system of FIG. 93.

FIG. 106 illustrates an environmental, rear view of the vest and bladder system of FIG. 93.

FIG. 107 illustrates an environmental, perspective, rear, right side view of the vest and bladder system of FIG. 93.

FIG. 108 illustrates a perspective, front view of another head and neck bladder device according to an aspect of the present disclosure.

FIG. 109 illustrates a perspective, rear view of the head and neck bladder device of FIG. 108.

FIG. 110 illustrates a perspective, rear, right side view of the head and neck bladder device of FIG. 108.

FIG. 111 illustrates an environmental, perspective, front, left side view of the head and neck bladder device of FIG. 108.

FIG. 112 illustrates a front view of a vest device according to an aspect of the present disclosure.

FIG. 113 illustrates a perspective, side view of the vest device of FIG. 112.

FIG. 114 illustrates a front, perspective view of a vest device according to an aspect of the present disclosure.

FIG. 115 illustrates a view of the vest device of FIG. 114.

FIG. 116 illustrates a view of a bladder device according to an aspect of the present disclosure.

FIG. 117 illustrates a view of the bladder device of FIG. 116.

FIG. 118 illustrates another view of the bladder device of FIG. 116.

FIG. 119 illustrates a further view of the bladder device of FIG. 116.

FIG. 120 illustrates another view of the bladder device of FIG. 116.

FIG. 121 illustrates a view of a bladder device according to an aspect of the present disclosure.

FIG. 122 illustrates a view of the bladder device of FIG. 121.

FIG. 123 illustrates another view of the bladder device of FIG. 121.

FIG. 124 is a front, elevational, environmental view of a garment or article of clothing or a compression garment or article of clothing according to the present invention, showing a long sleeve shirt;

FIG. 125 is a front, elevational, environmental view of a garment or article of clothing or a compression garment or article of clothing according to the present invention, showing a short sleeve shirt;

FIG. 126 is a front, elevational, environmental view of a garment or article of clothing or a compression garment or article of clothing according to the present invention, showing a sleeveless shirt;

FIG. 127 is a front, perspective view of a garment or article of clothing or a compression garment or article of clothing according to the present invention, showing a pant;

FIG. 128 is a front, elevational view of a garment or article of clothing or a compression garment or article of clothing according to the present invention, showing a sweatshirt;

FIG. 129 is a front, elevational view of a garment or article of clothing or a compression garment or article of clothing according to the present invention, showing a jacket;

FIG. 130 is a side, elevational view of a garment or article of clothing or a compression garment or article of clothing according to the present invention, showing an arm sleeve;

FIG. 131 is a side, elevational, environmental view of a garment or article of clothing or a compression garment or article of clothing according to the present invention, showing a sock;

FIG. 132 is a front, elevational view of a compression garment or article of clothing according to the present invention, showing a compression vest (in a closed position), an inlet, an outlet, and connecting hoses;

FIG. 133 is a rear, elevational view of the compression garment or article of clothing of FIG. 132;

FIG. 134 is an elevational view of the compression garment or article of clothing of FIG. 132, showing the compression vest in an open position;

FIG. 135 is a front, elevational view of an air bladder system according to the present invention.

It should be understood that the above-attached figures are not intended to limit the scope of the present invention in any way.

### DETAILED DESCRIPTION OF THE PREFERRED EMBODIMENTS

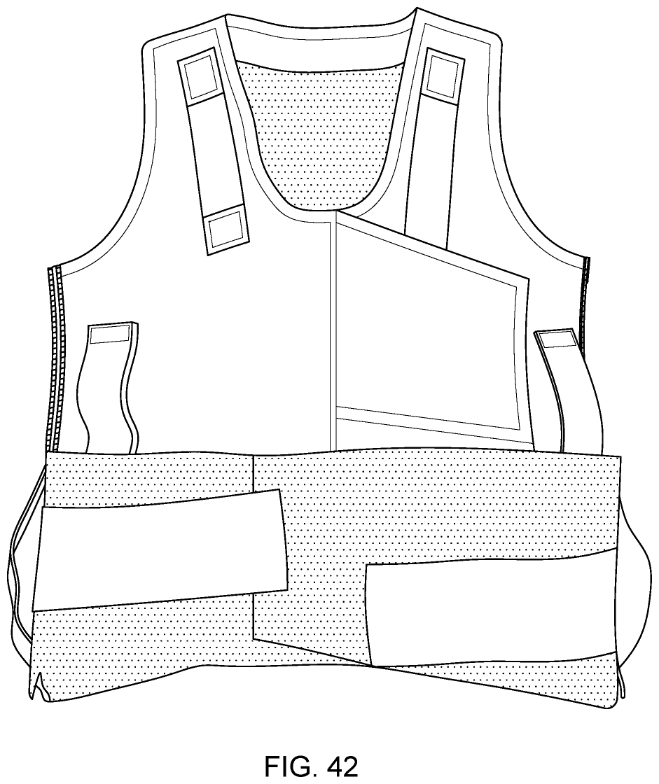

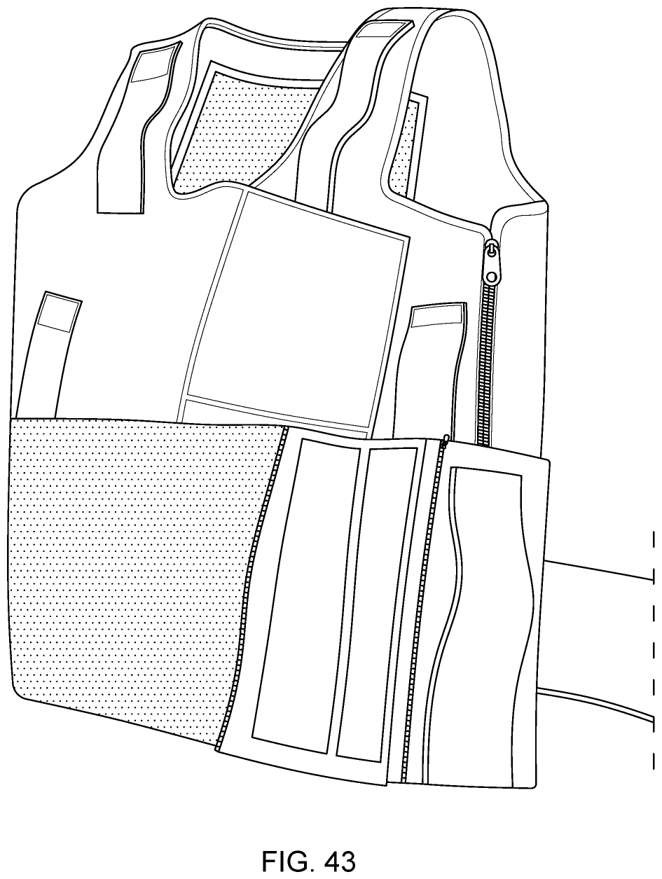

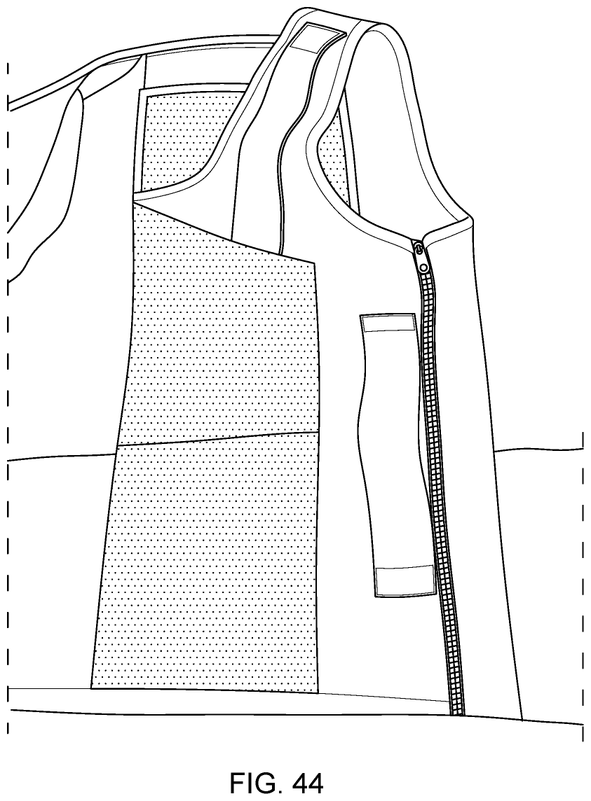

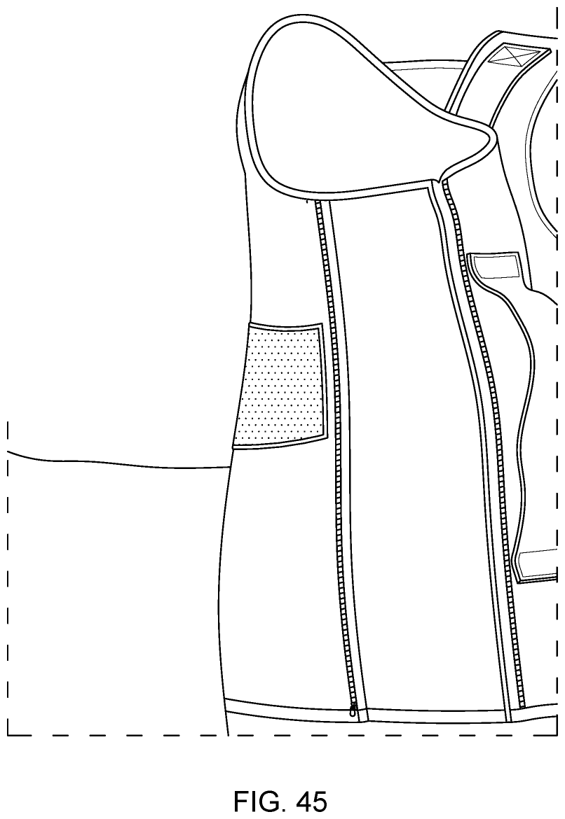

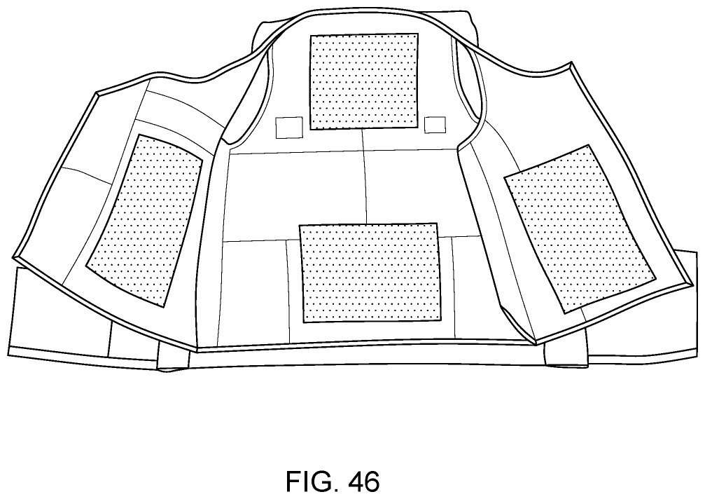

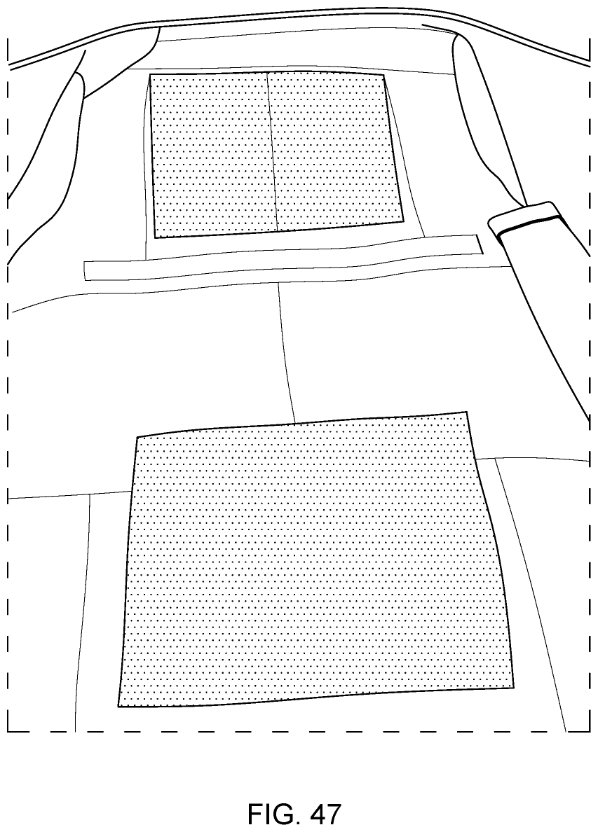

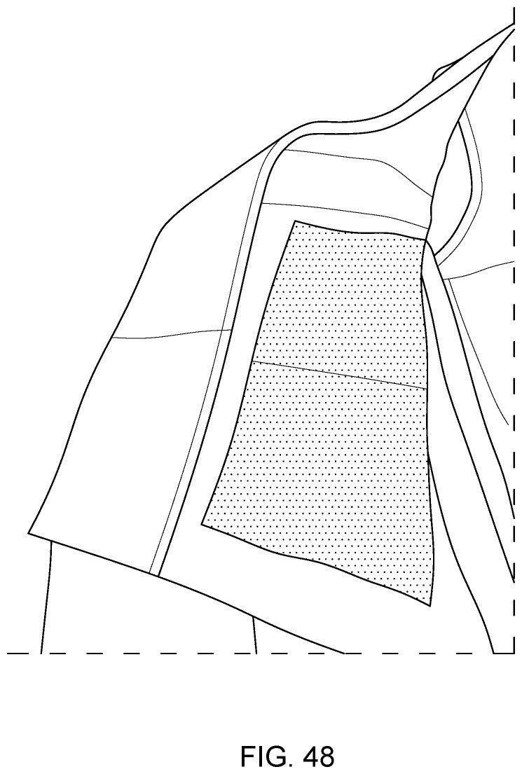

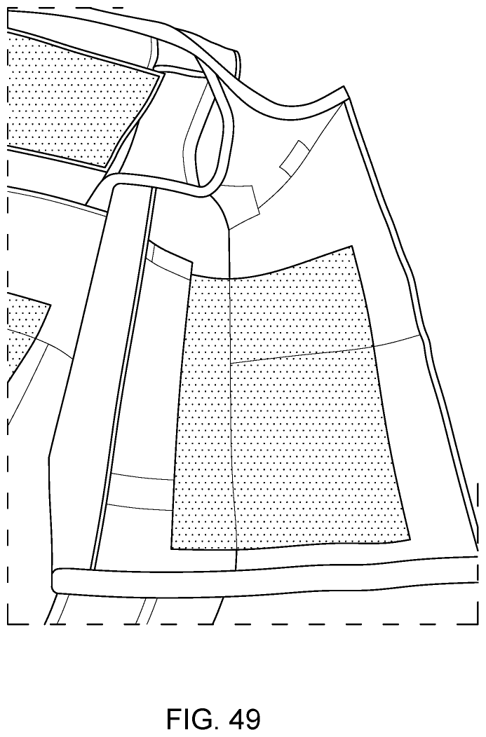

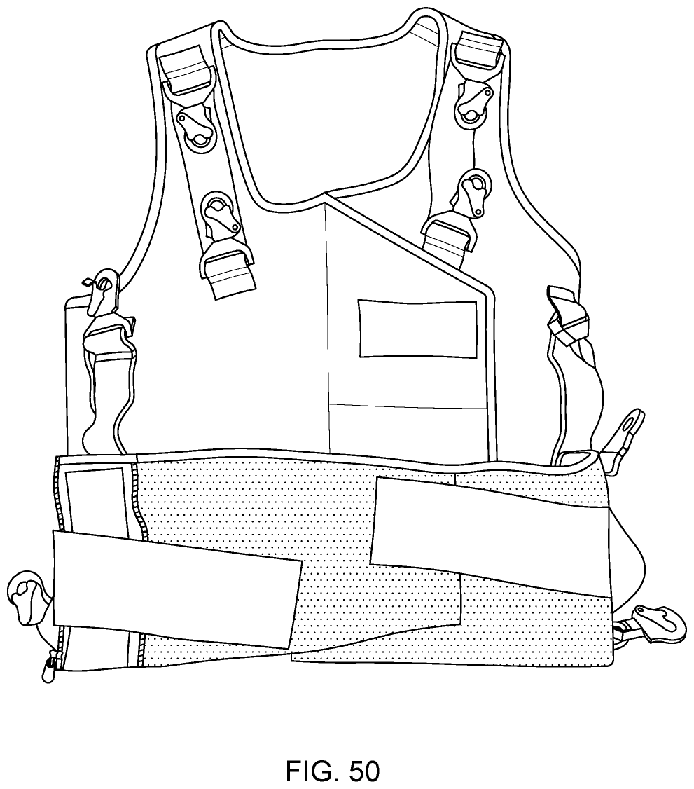

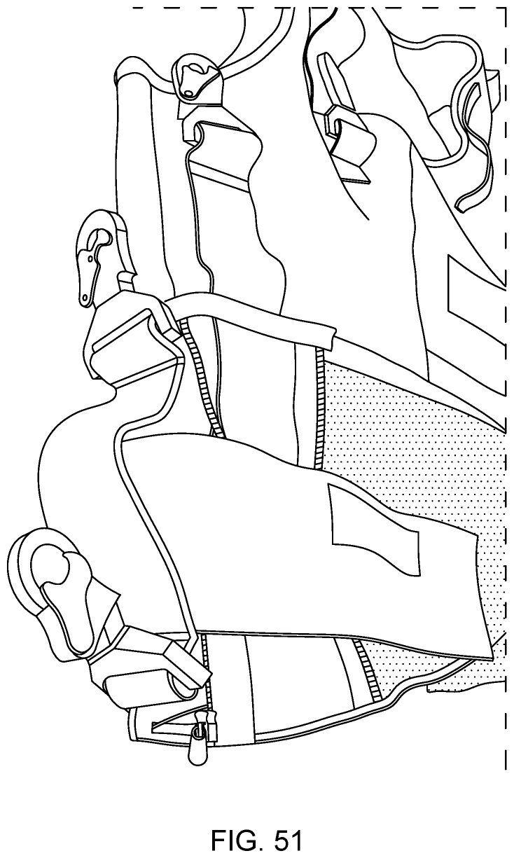

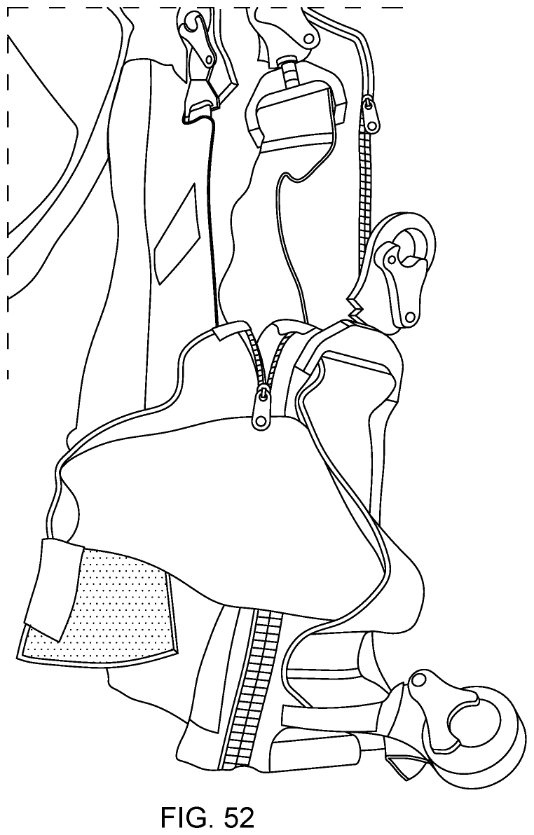

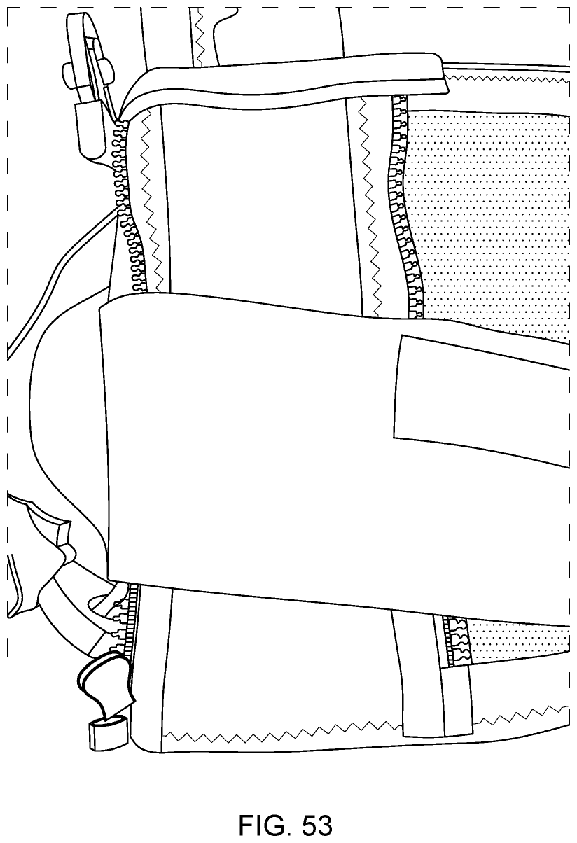

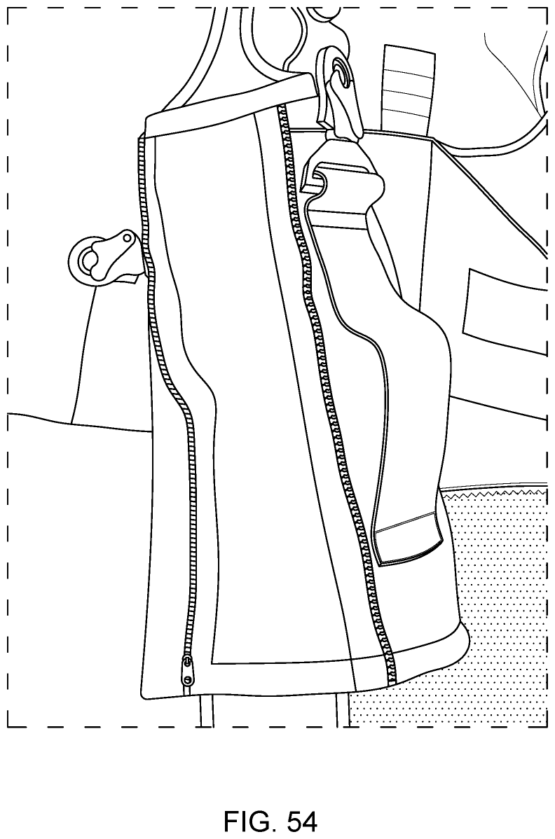

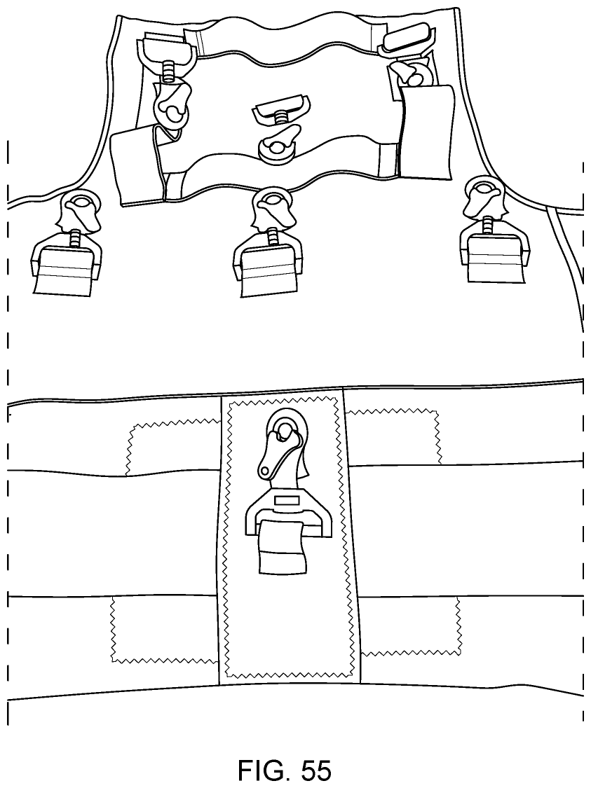

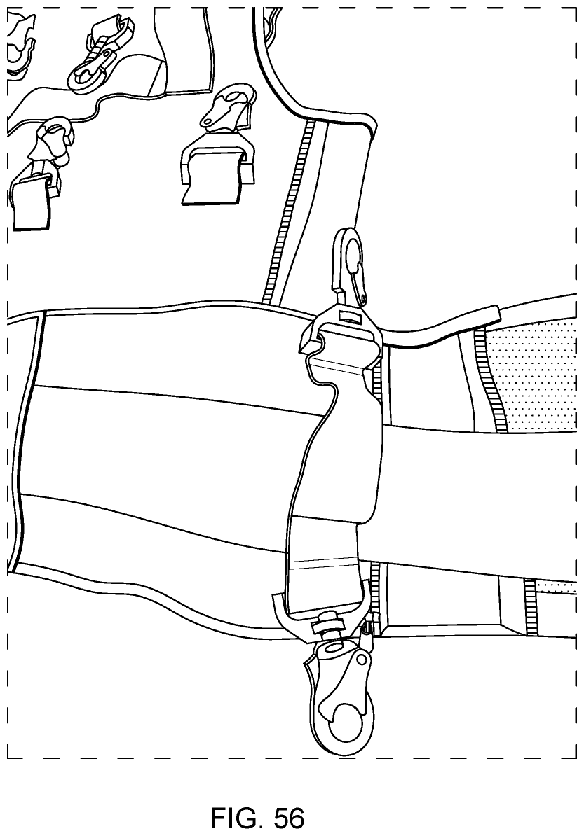

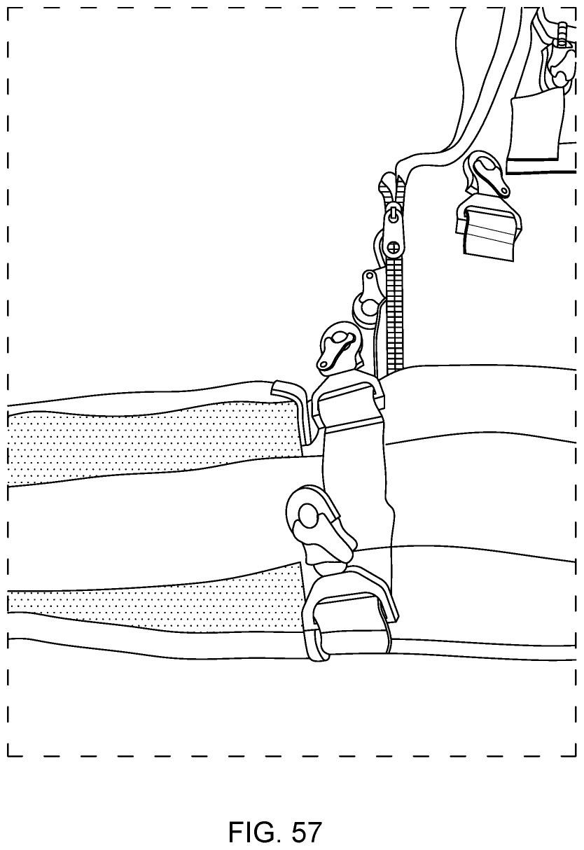

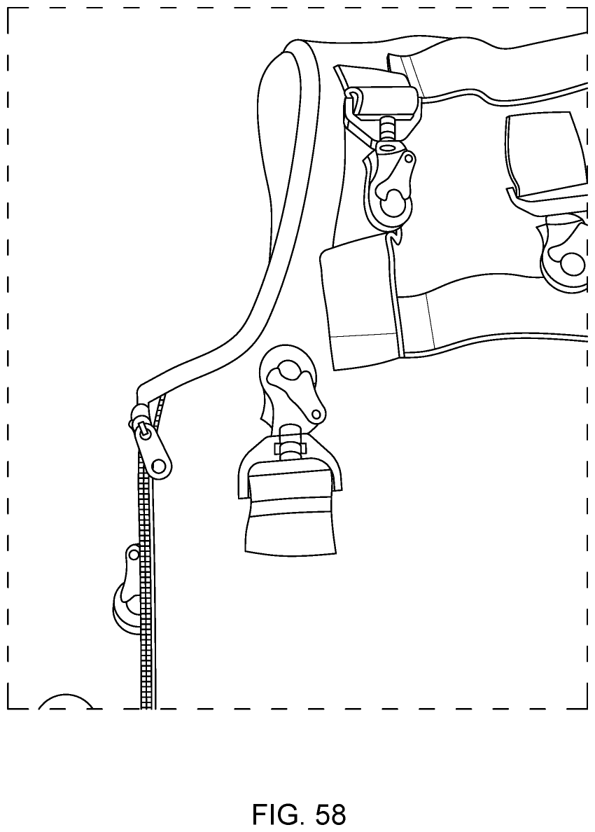

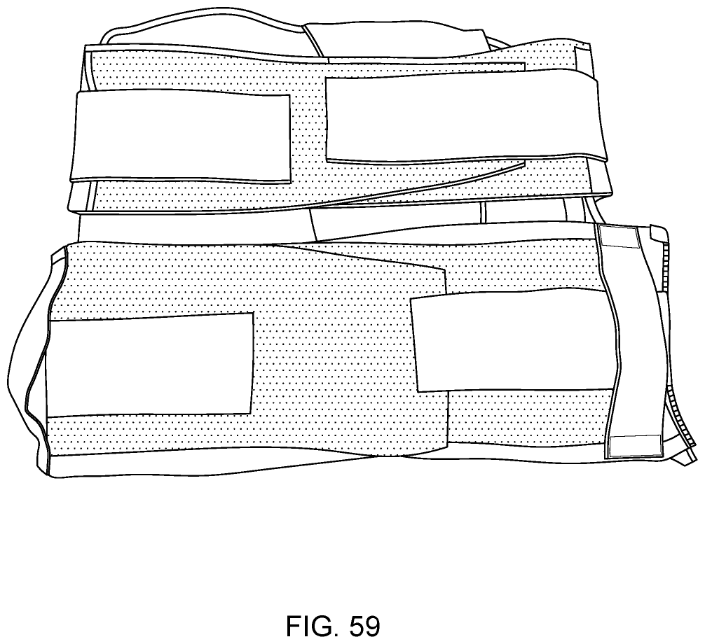

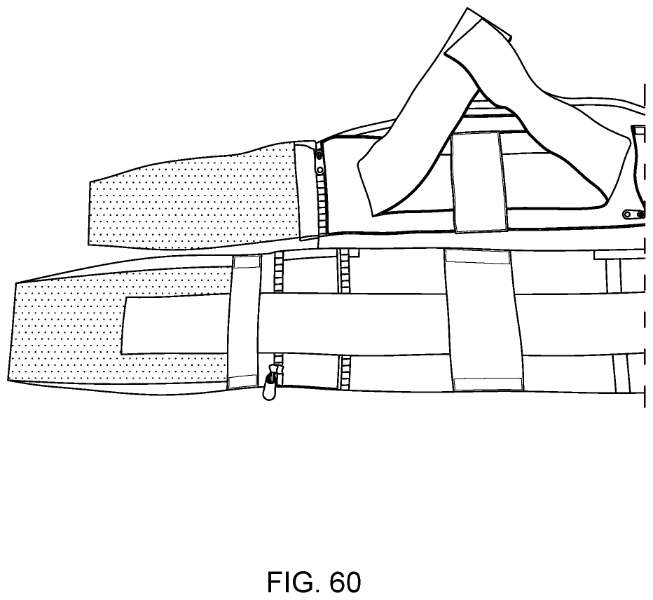

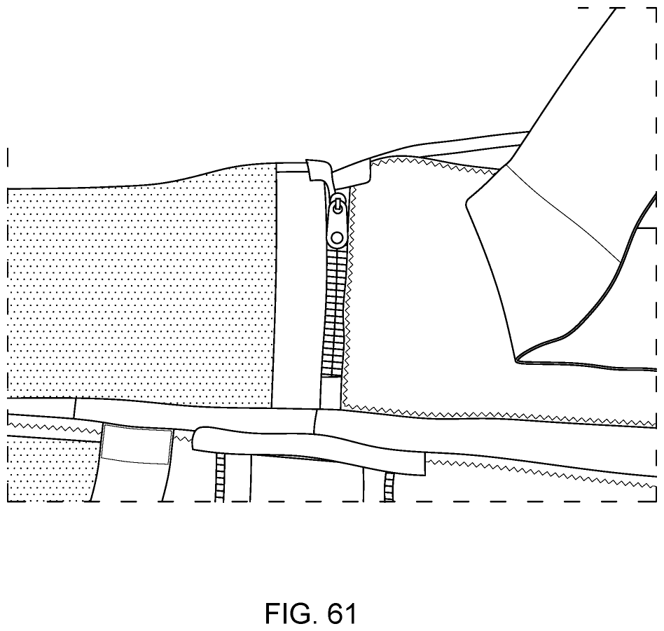

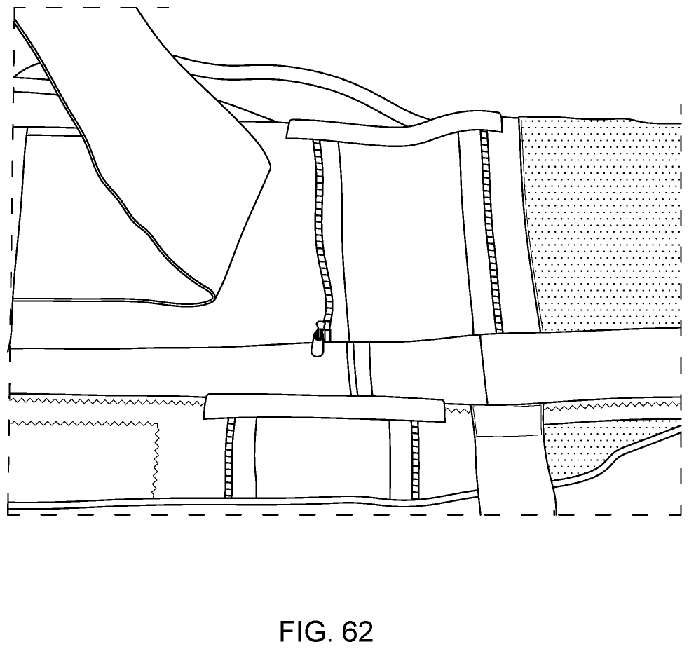

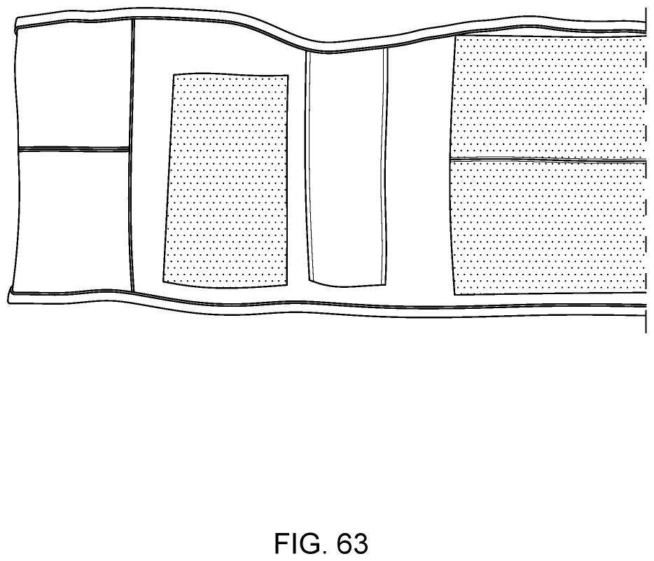

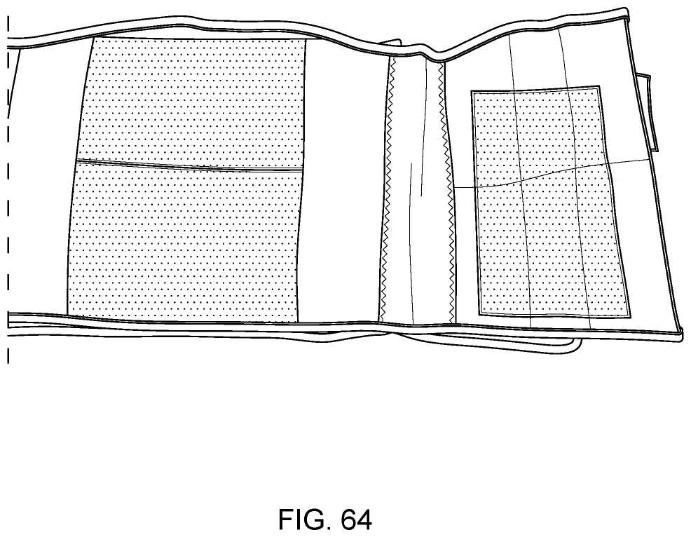

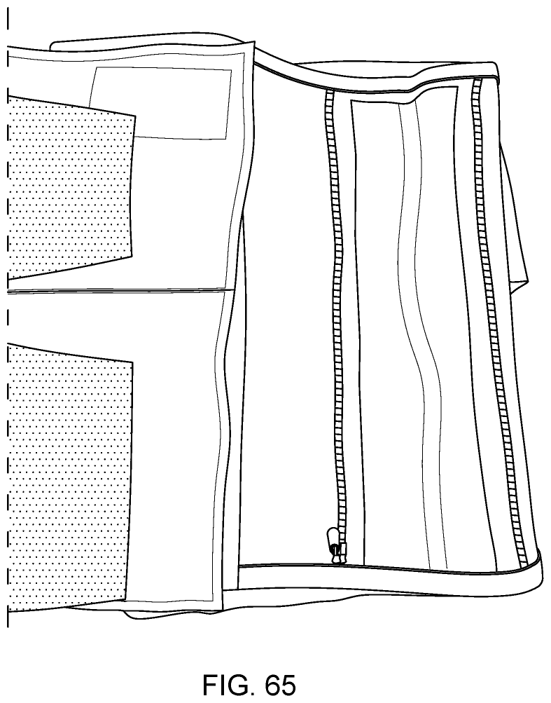

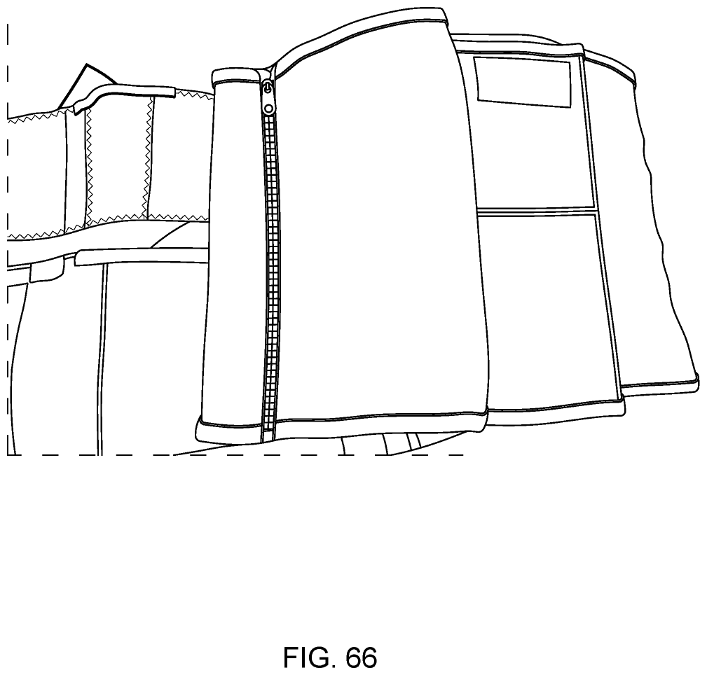

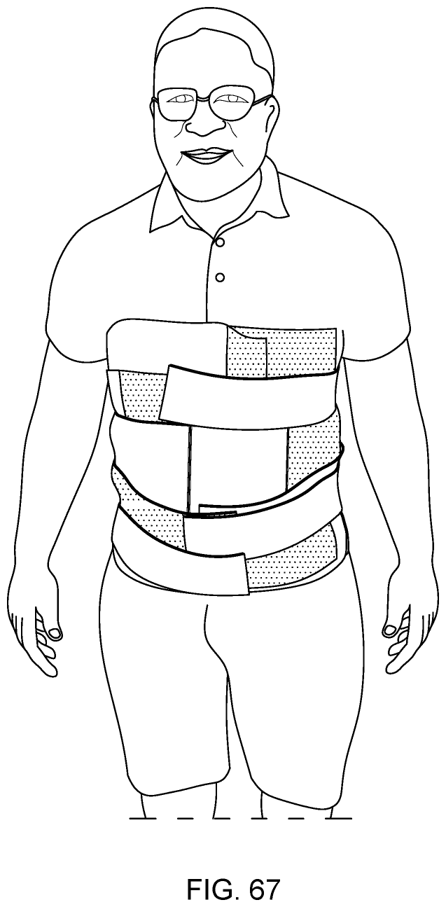

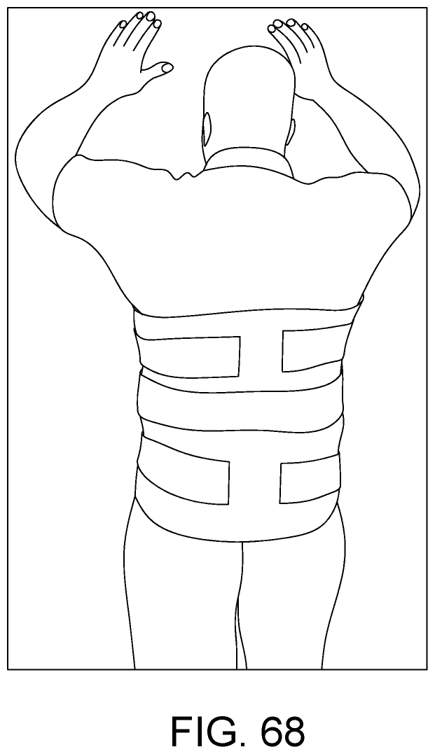

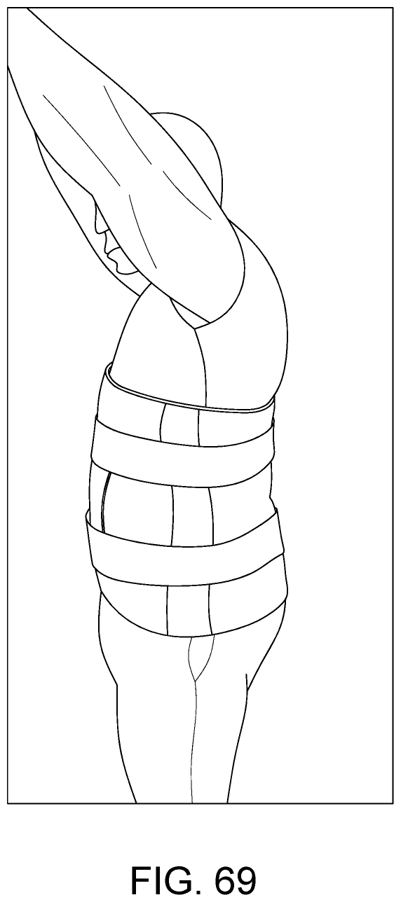

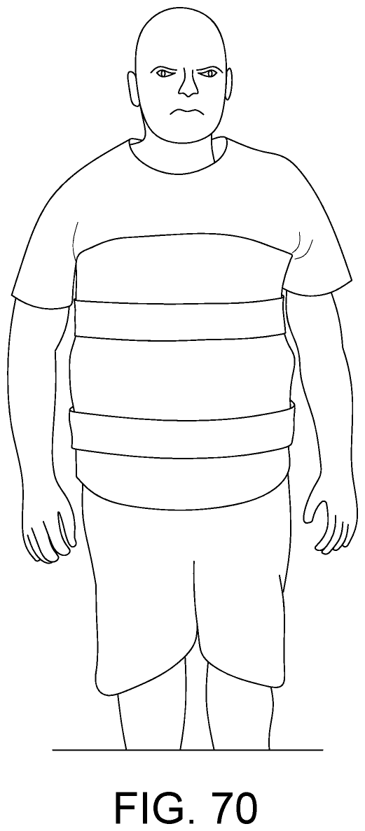

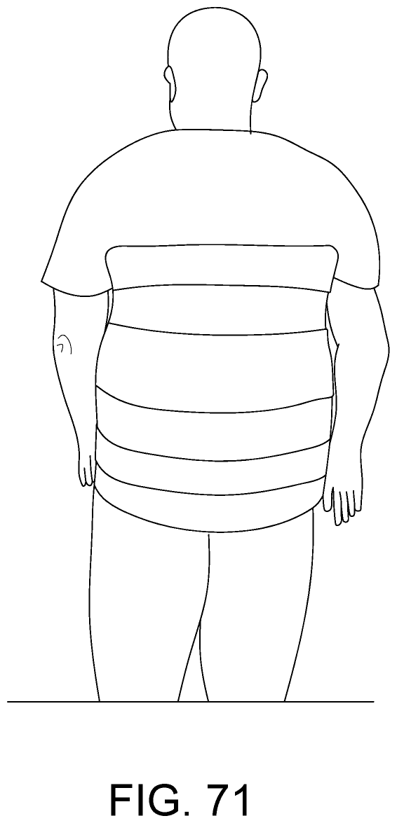

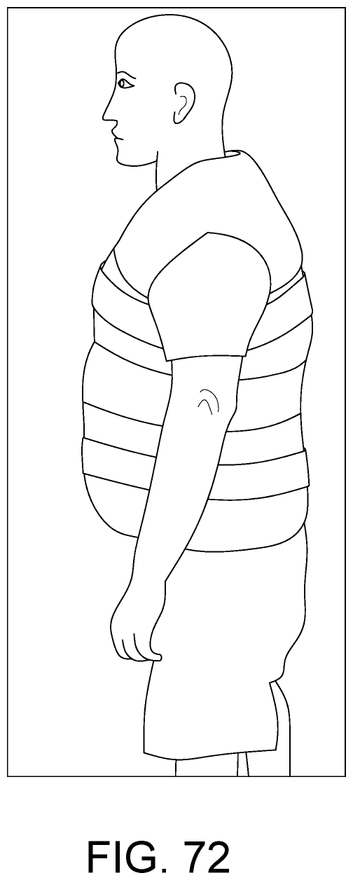

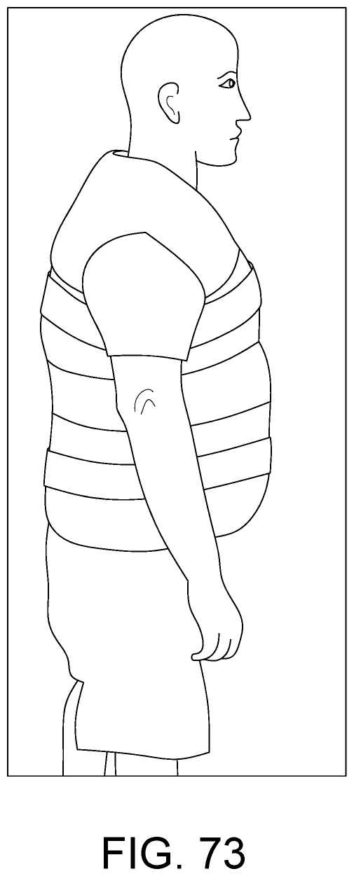

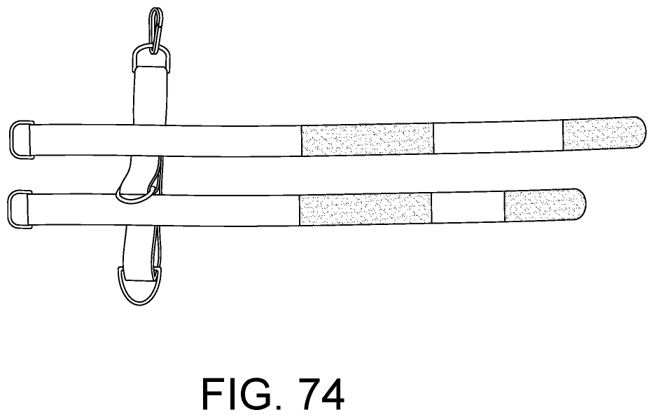

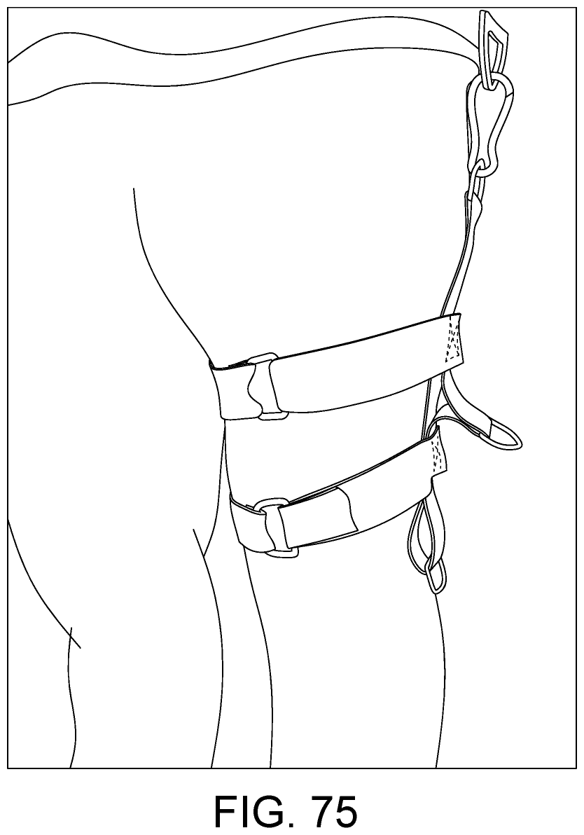

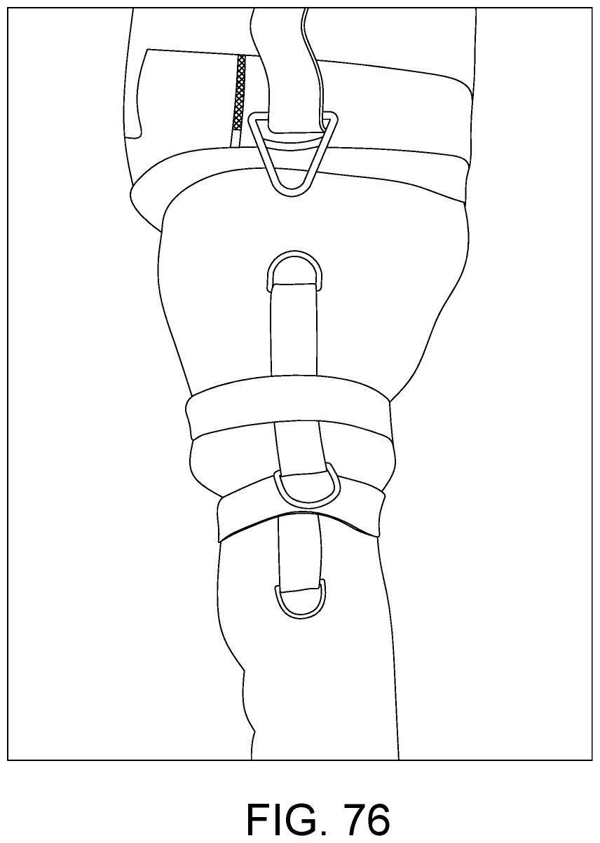

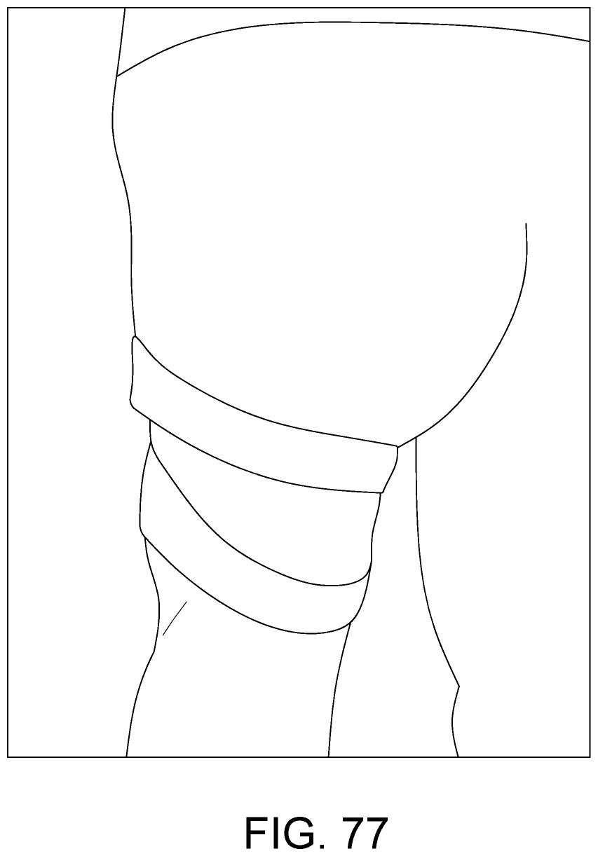

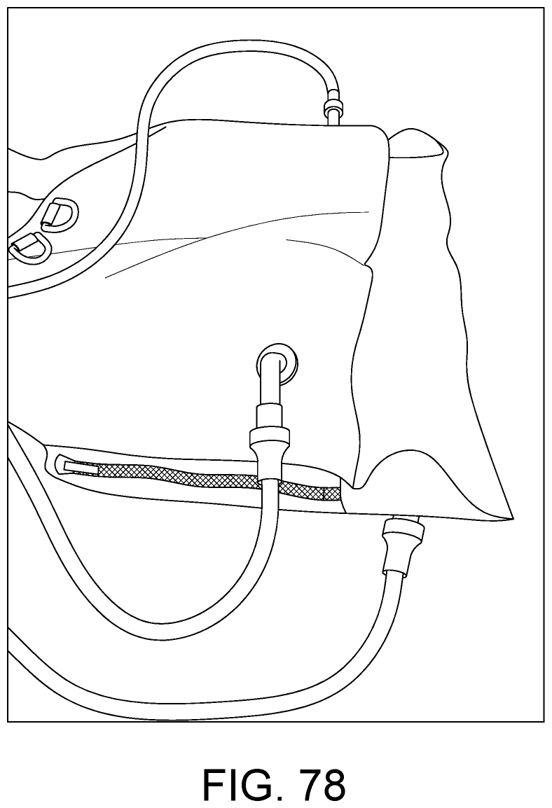

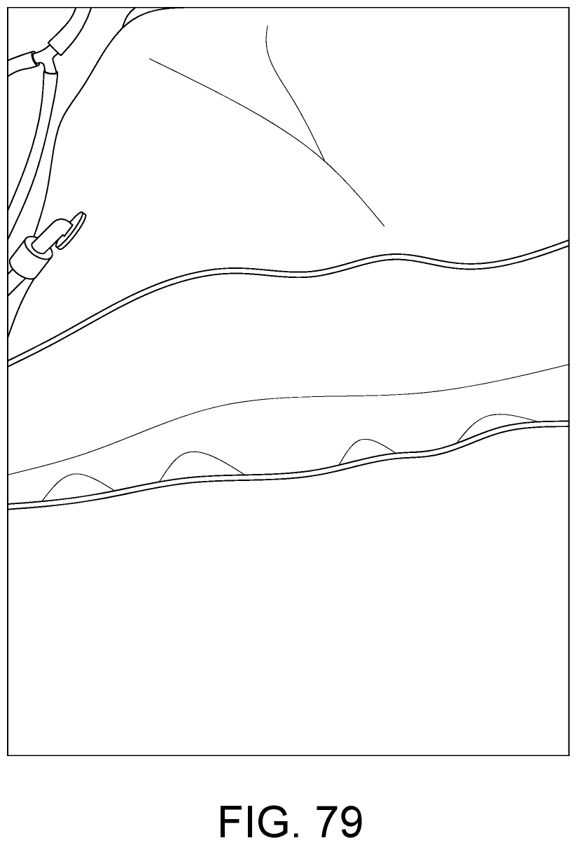

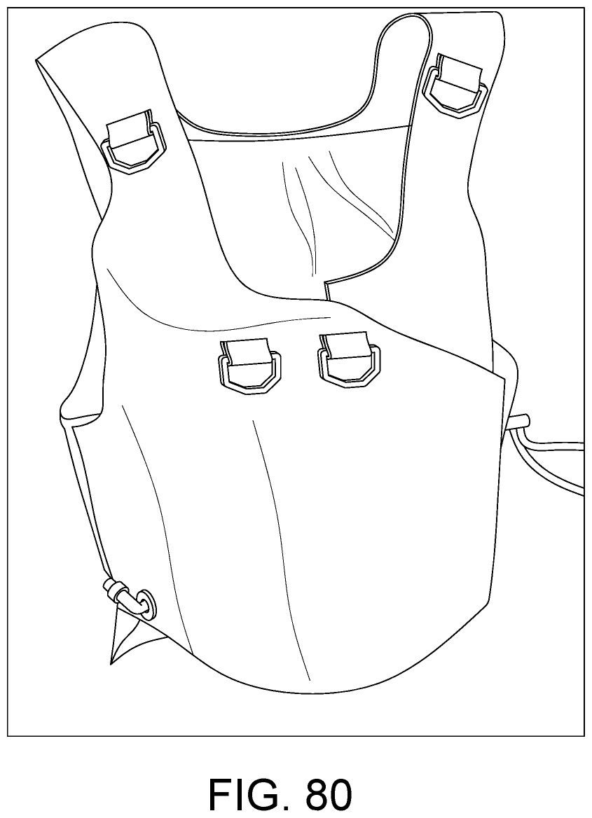

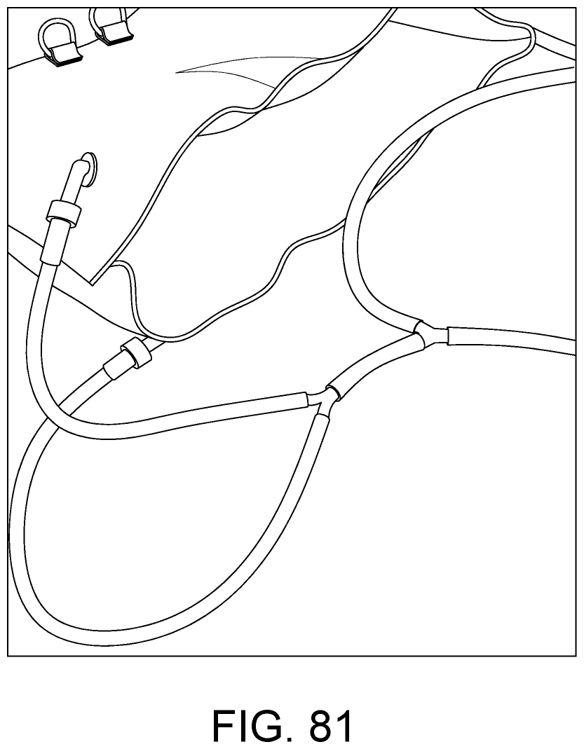

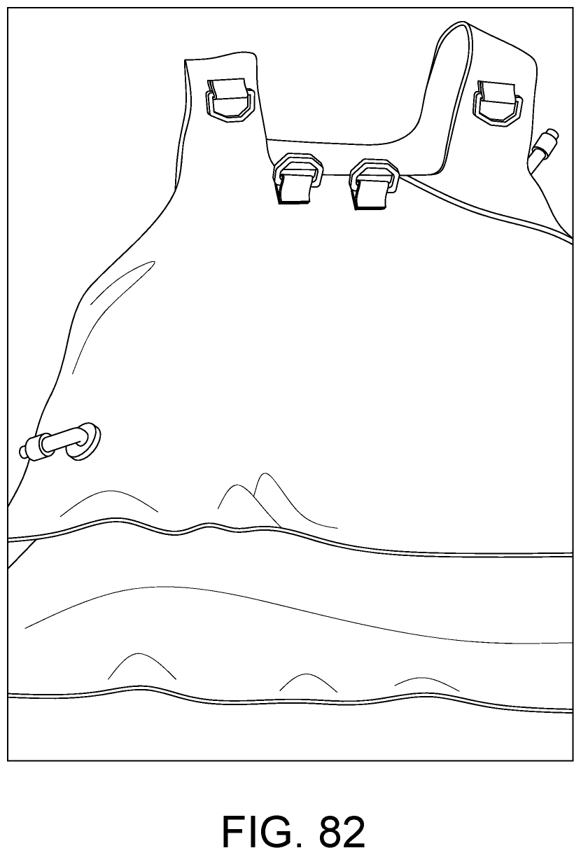

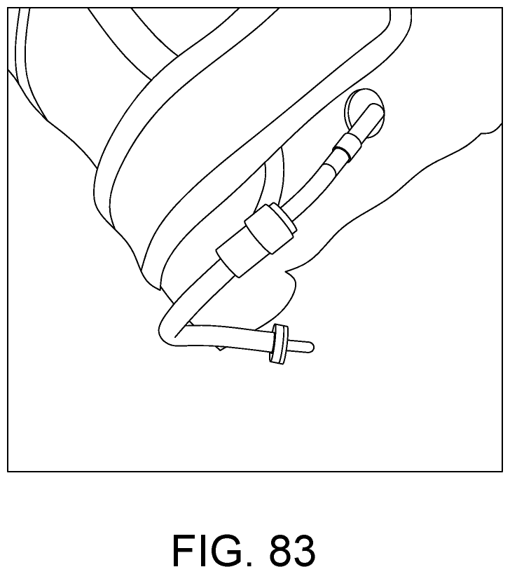

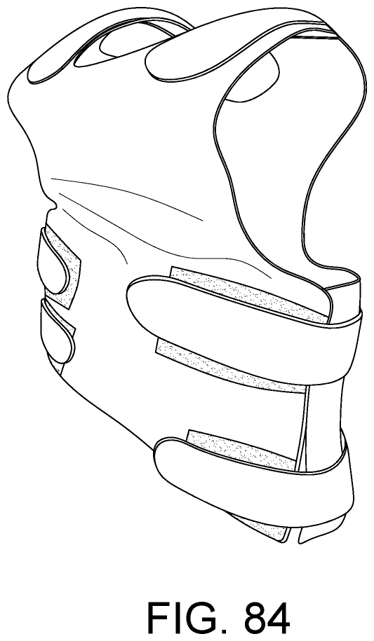

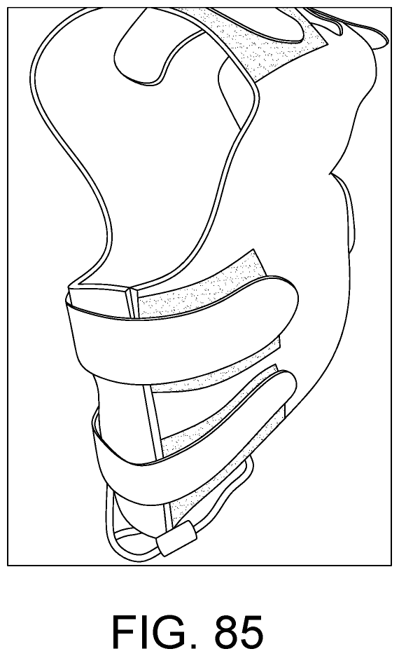

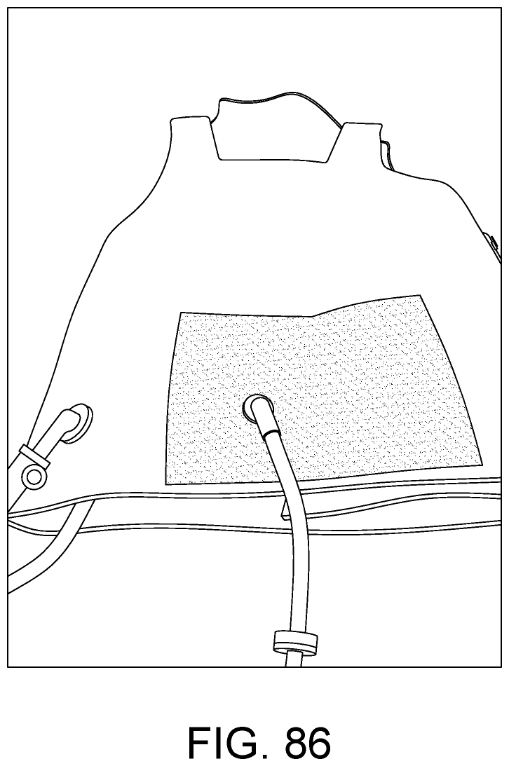

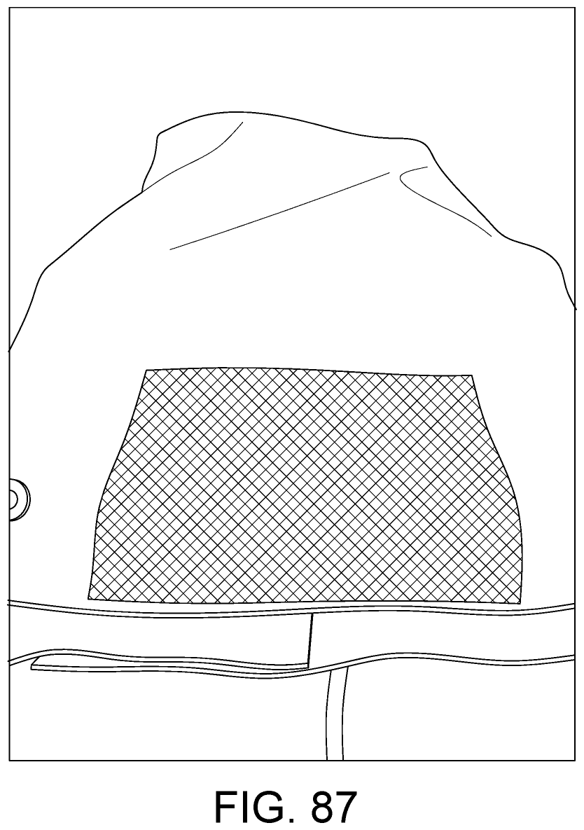

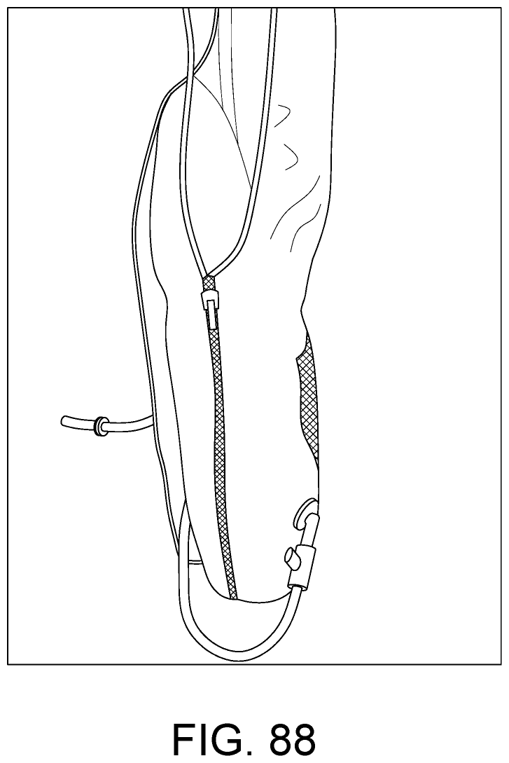

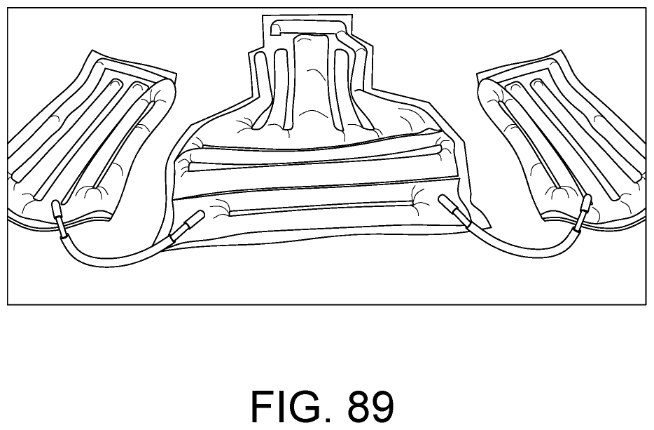

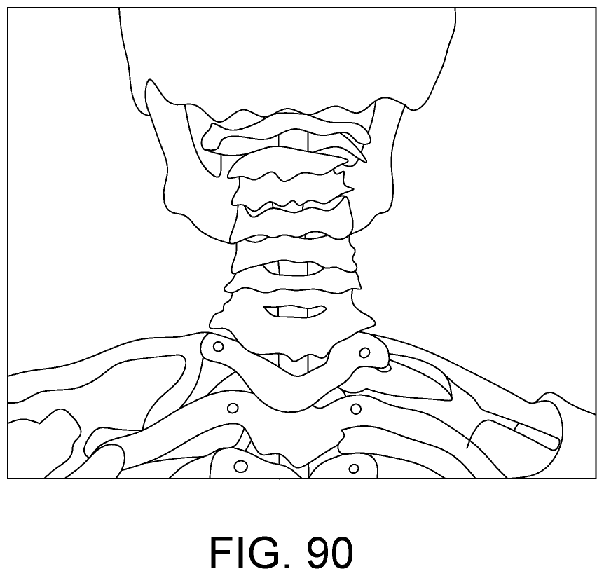

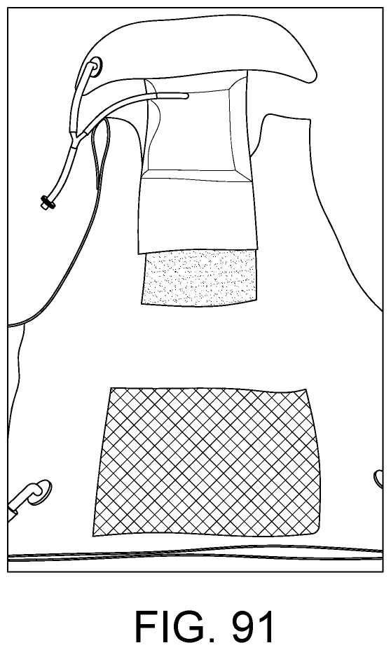

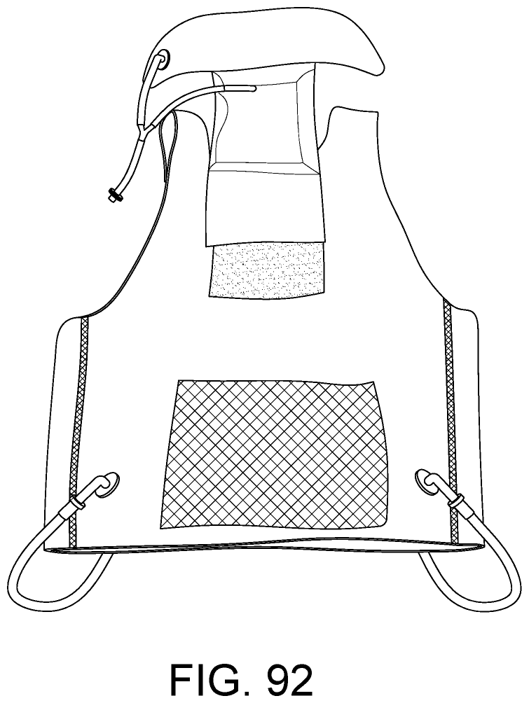

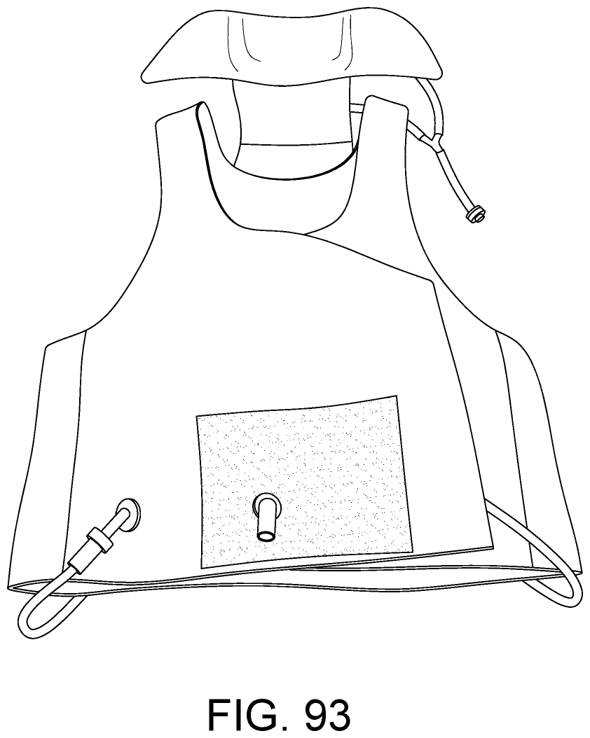

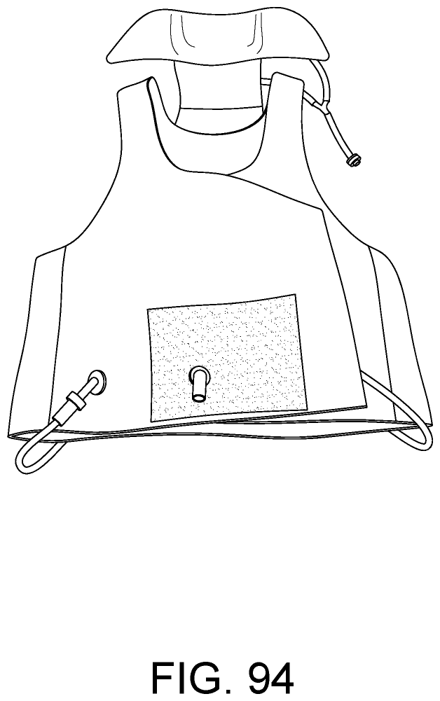

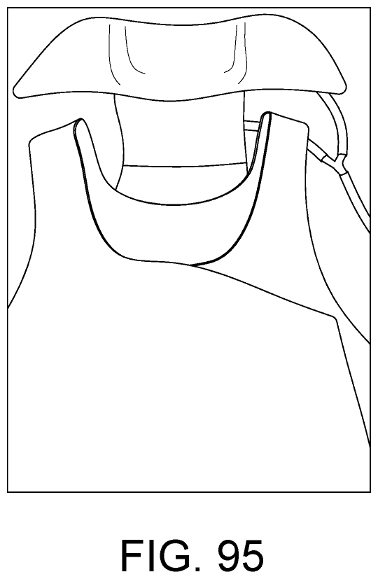

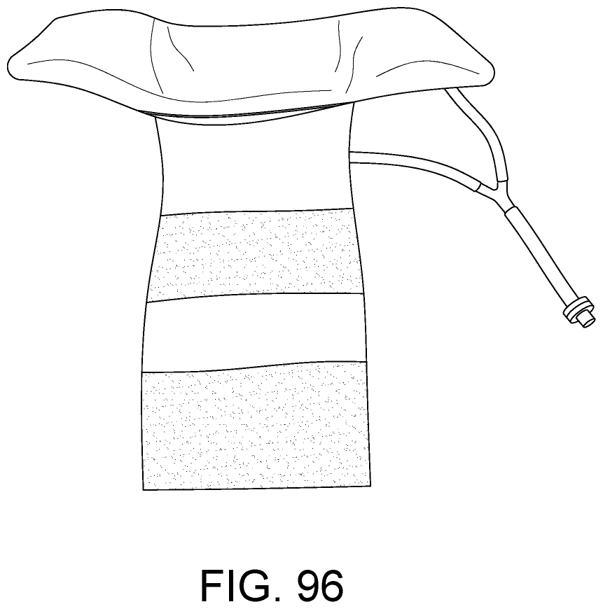

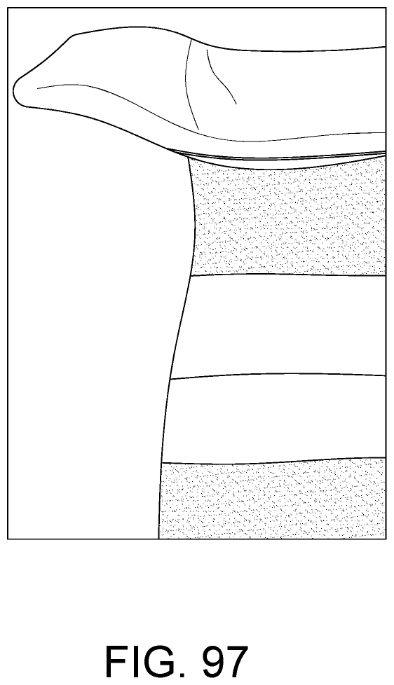

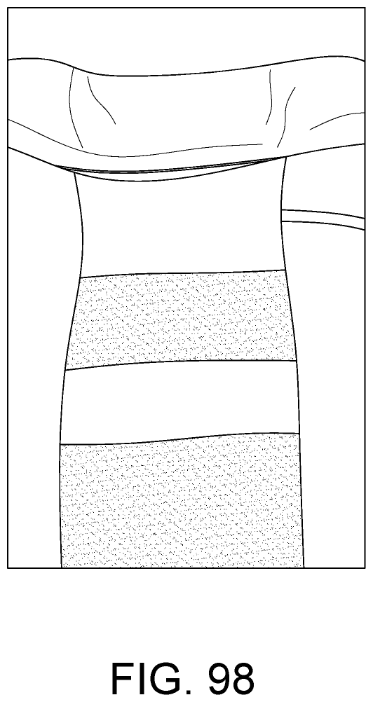

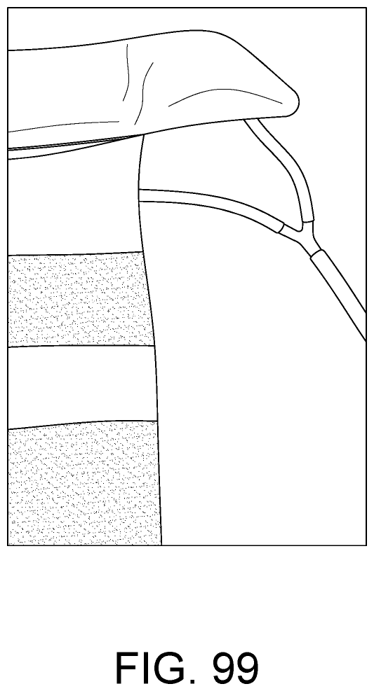

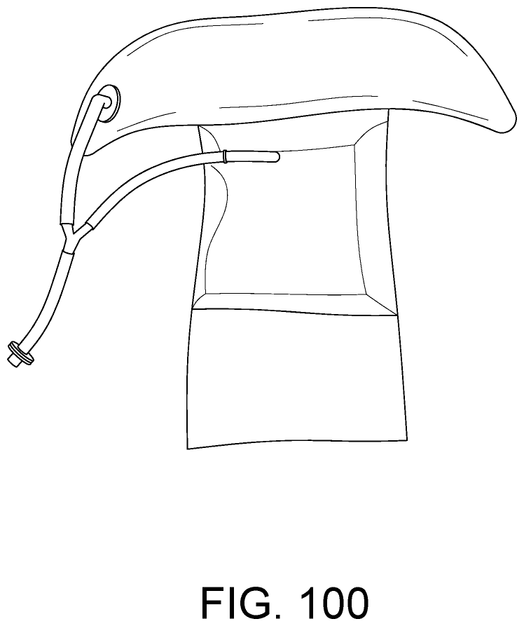

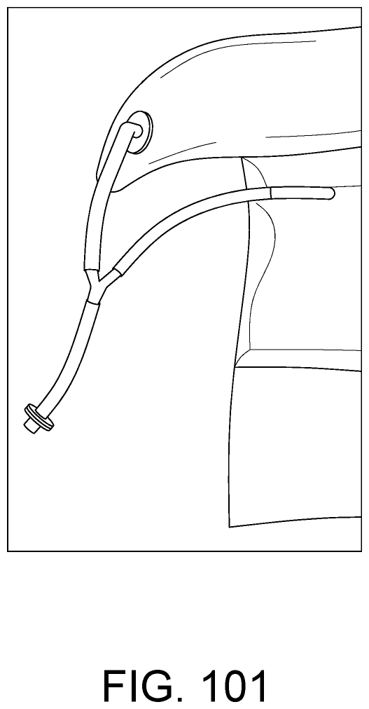

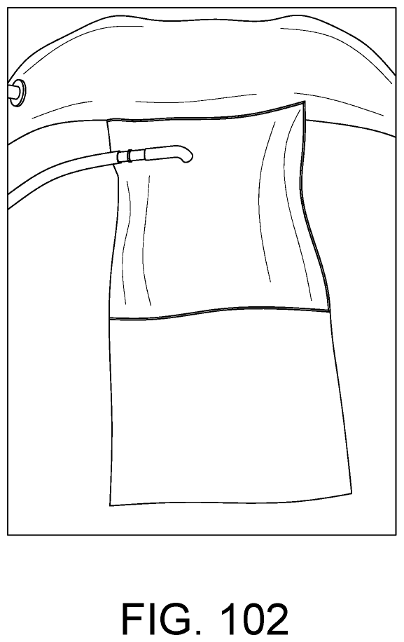

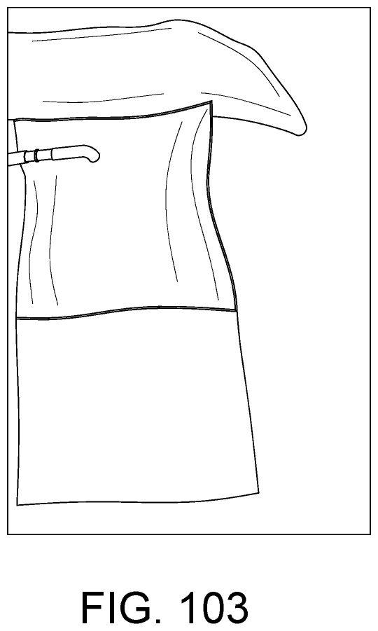

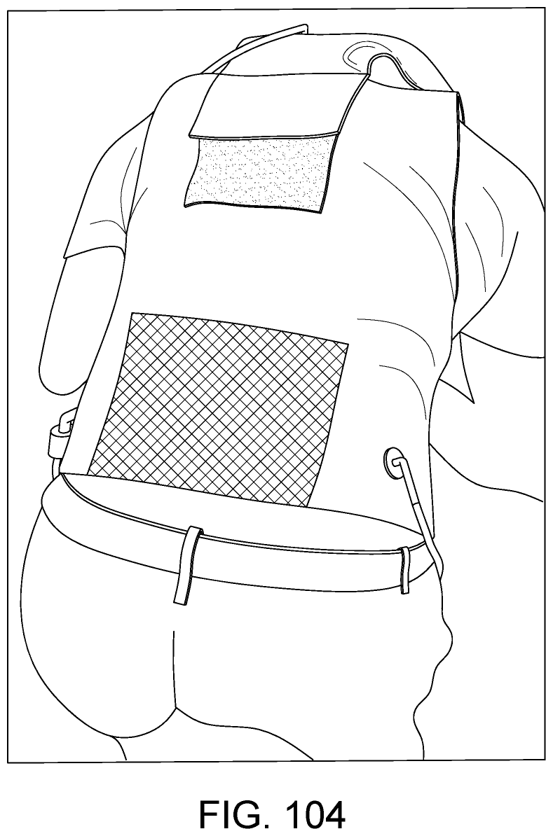

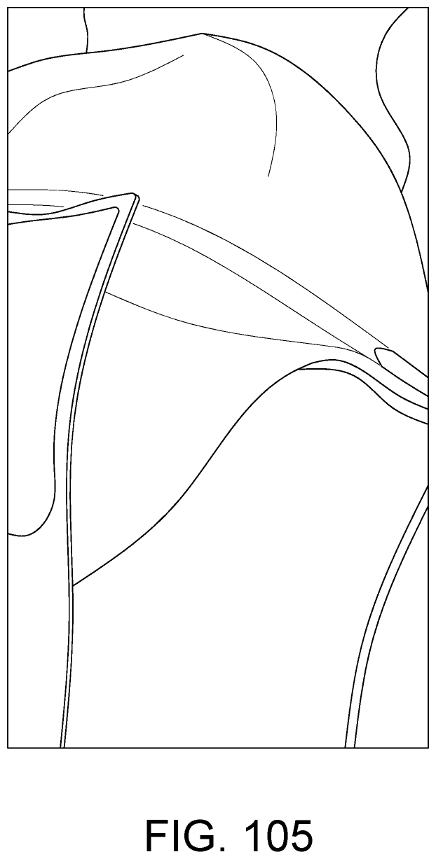

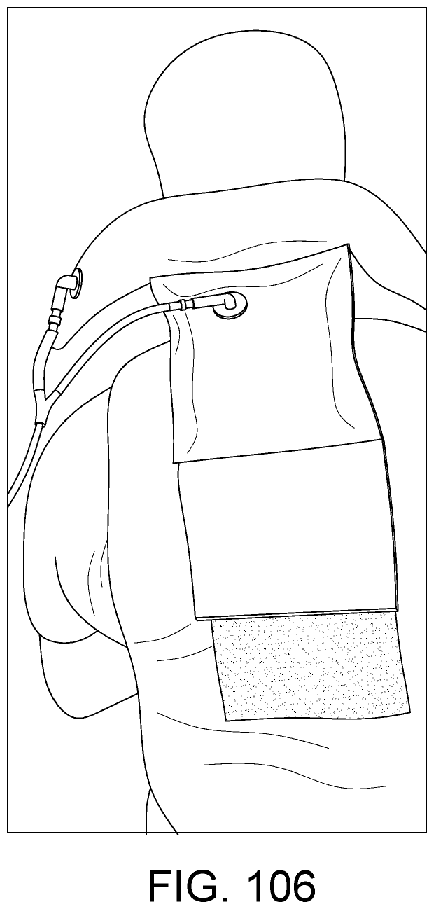

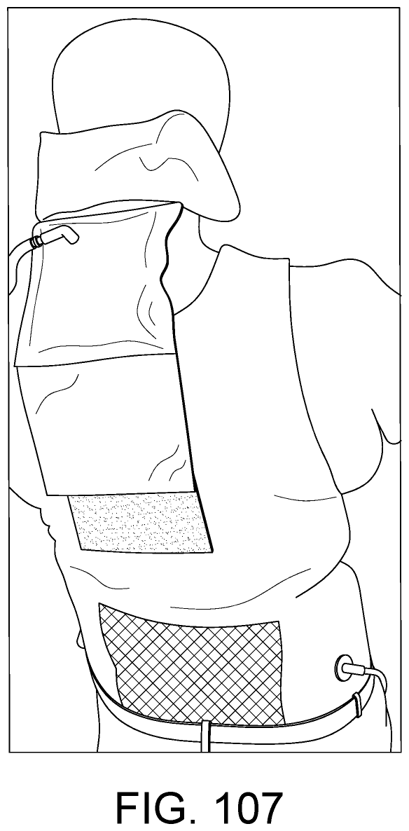

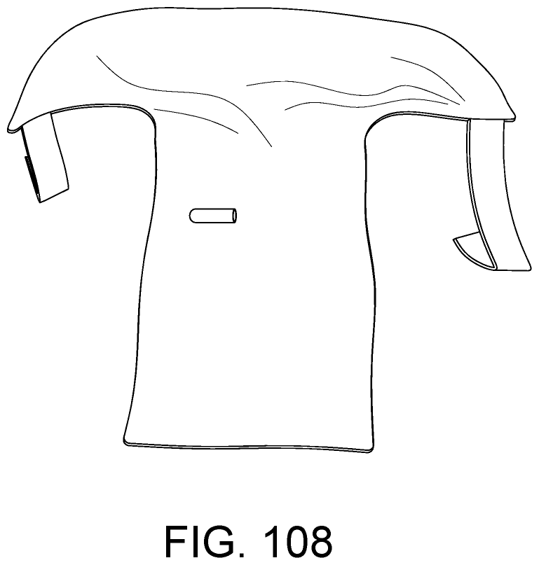

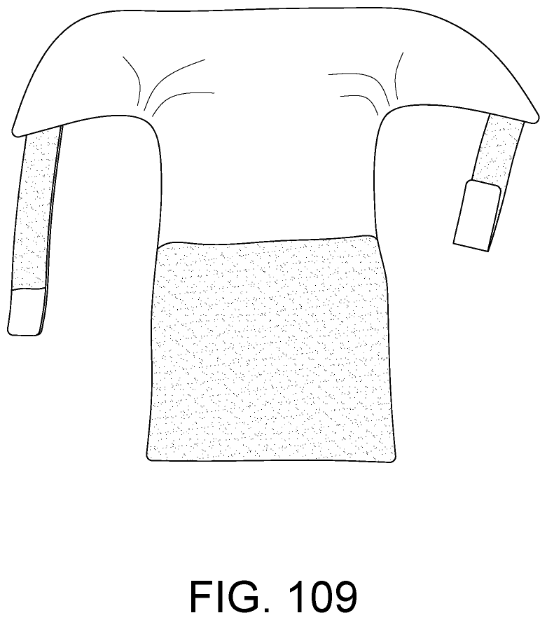

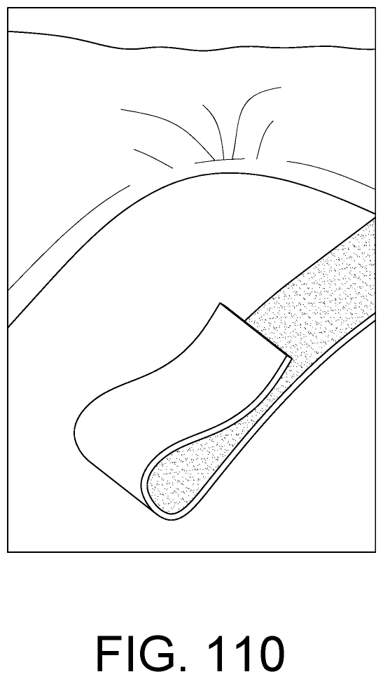

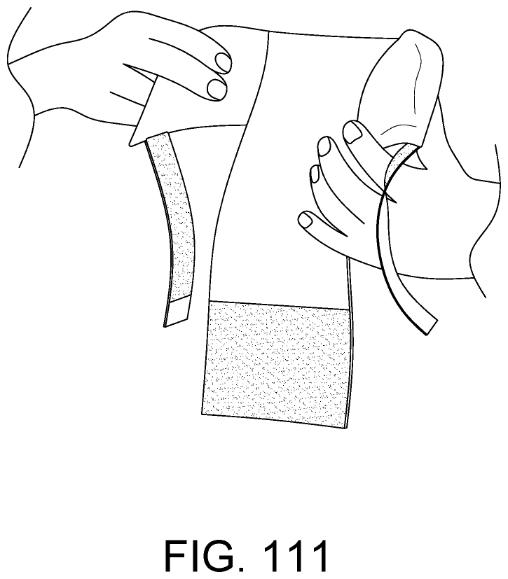

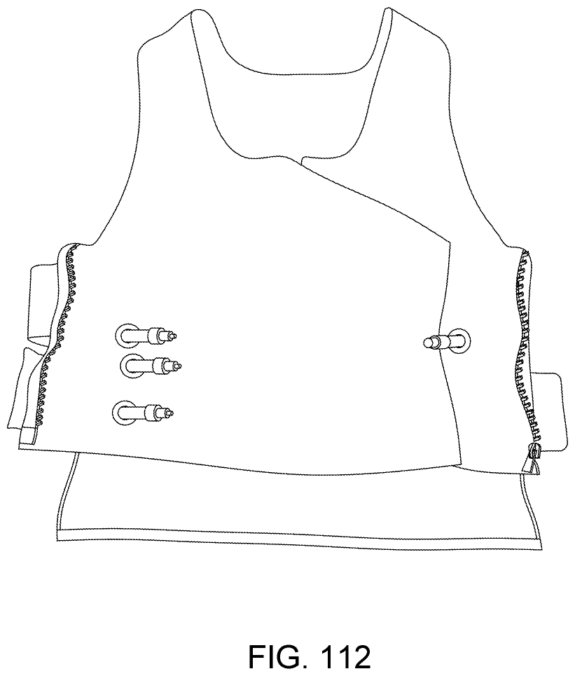

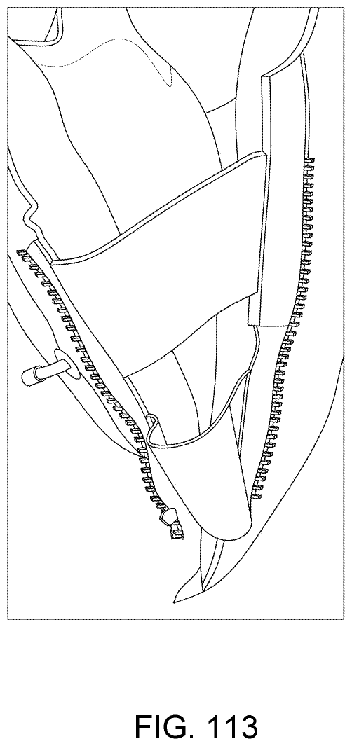

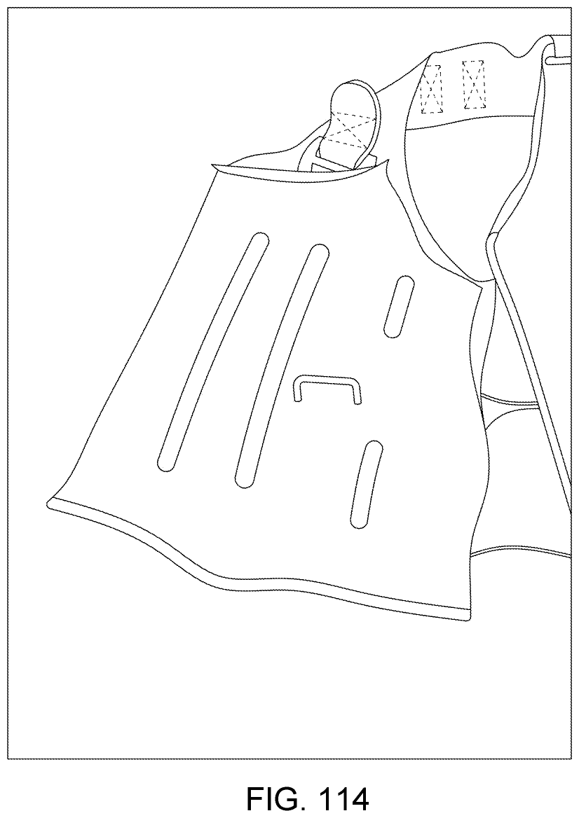

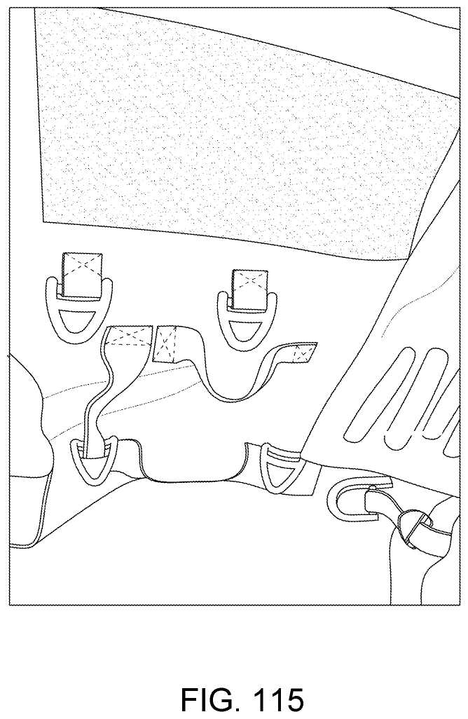

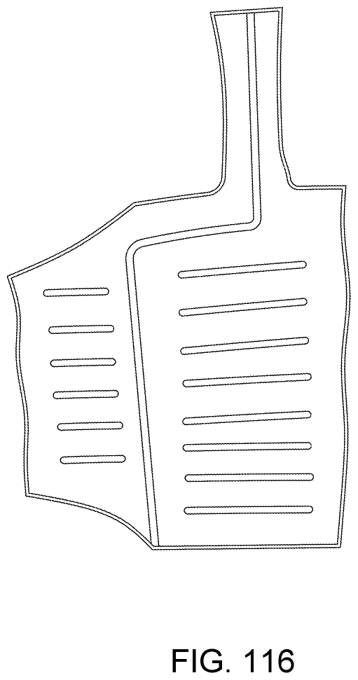

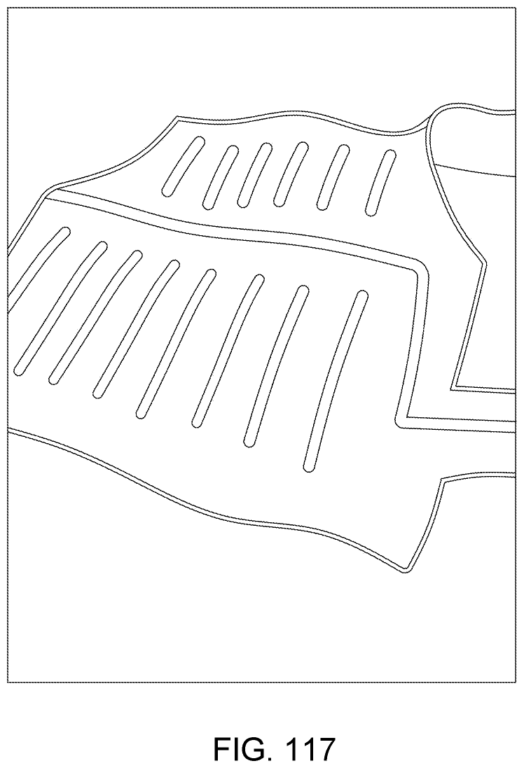

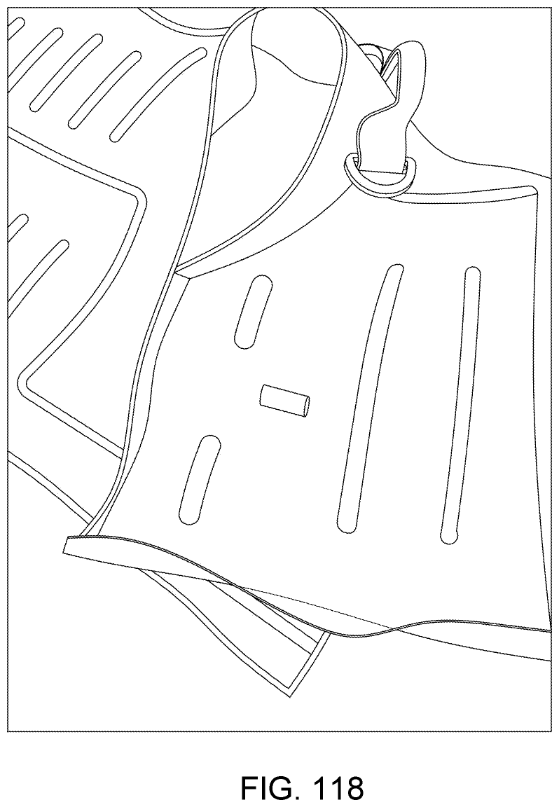

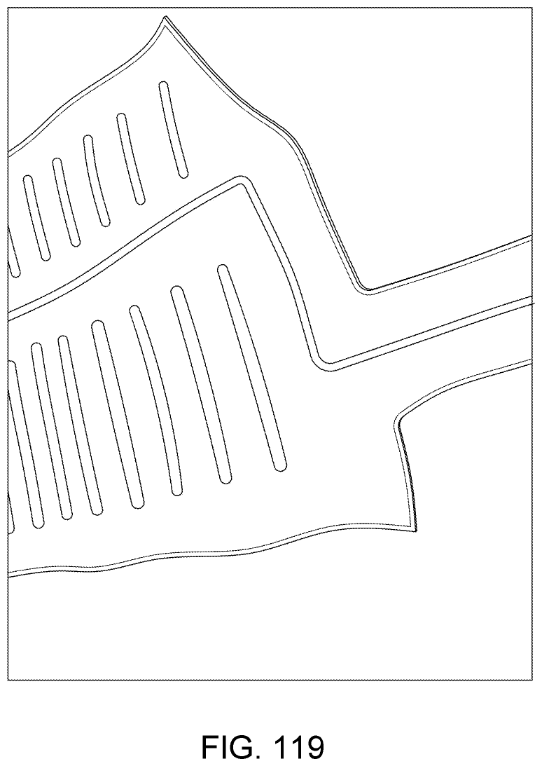

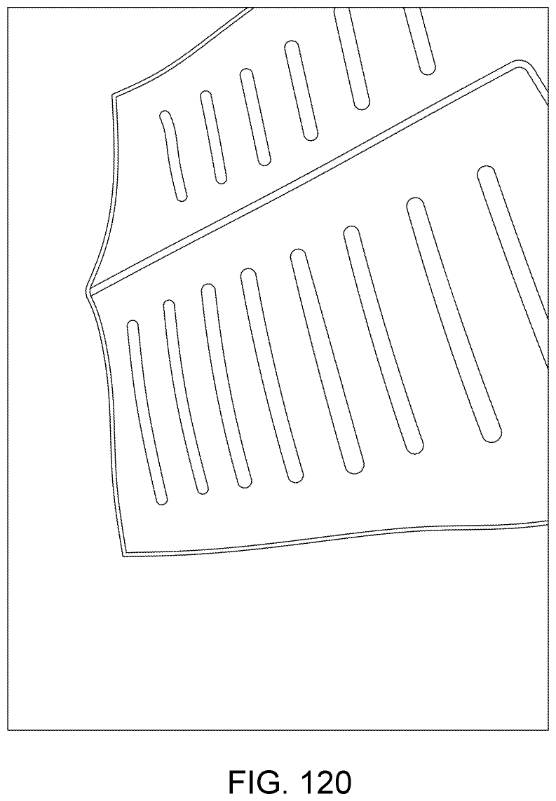

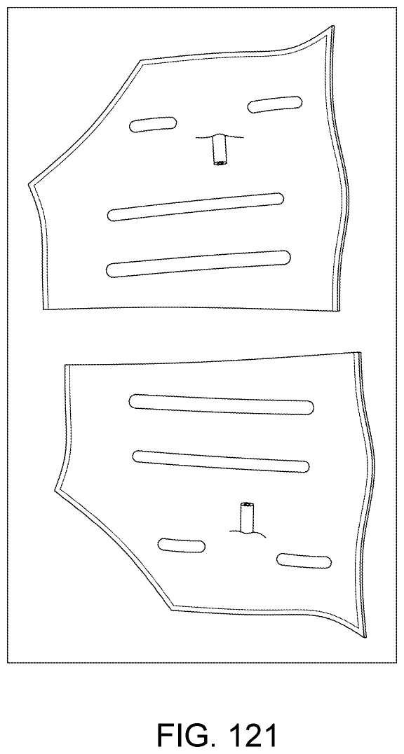

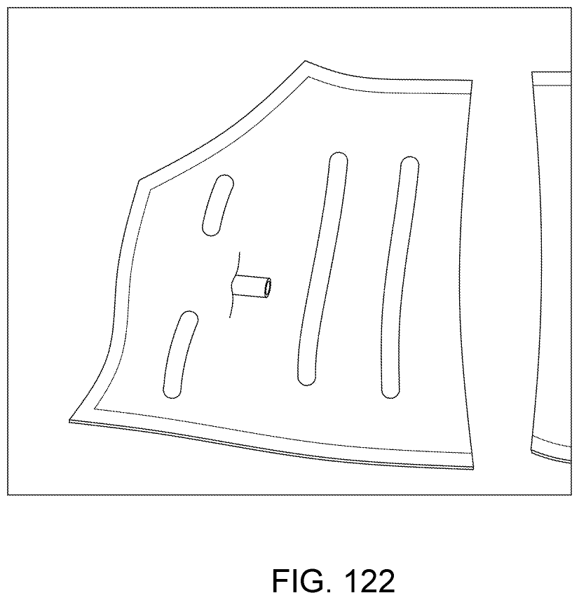

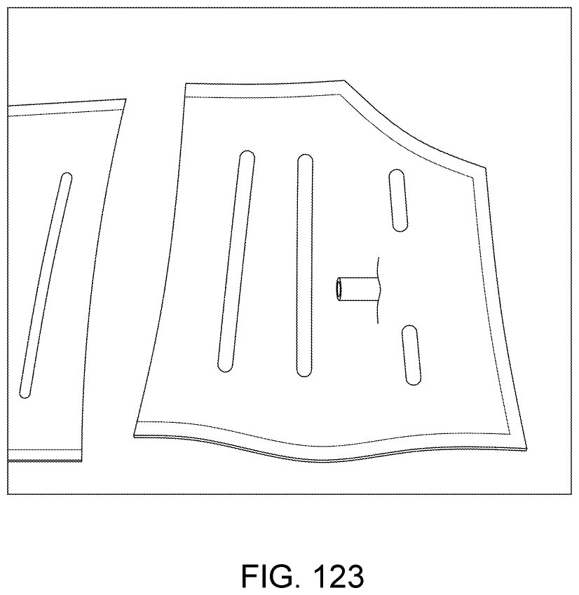

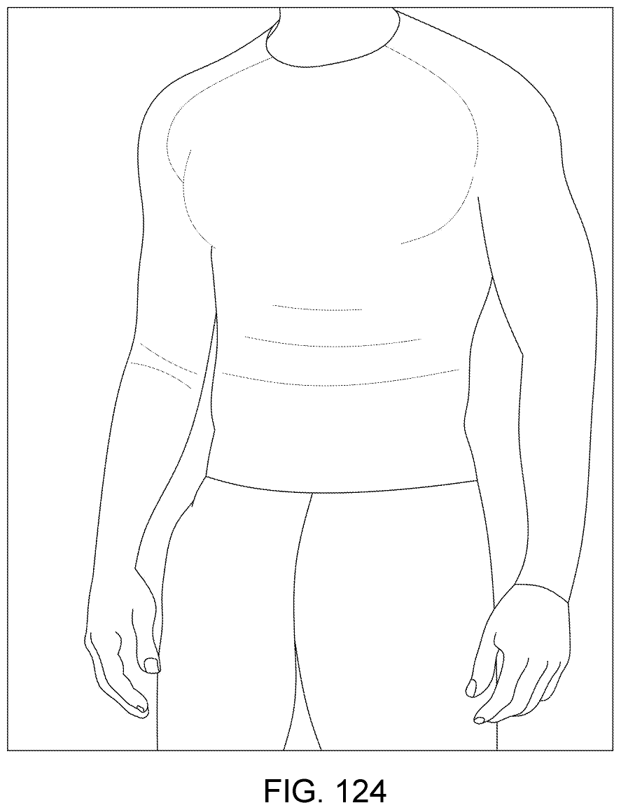

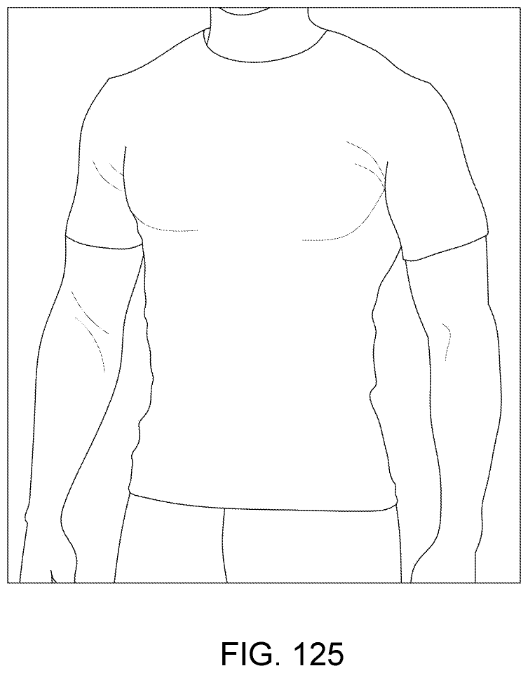

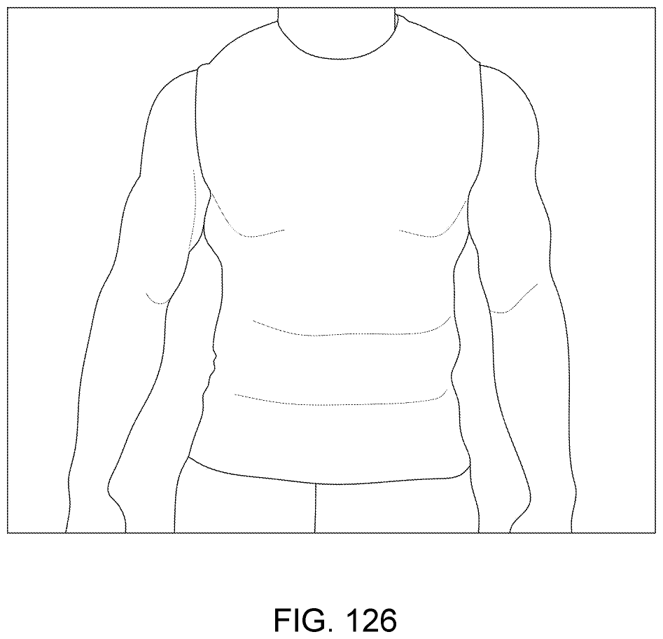

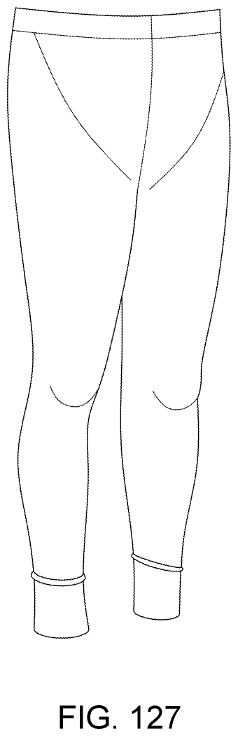

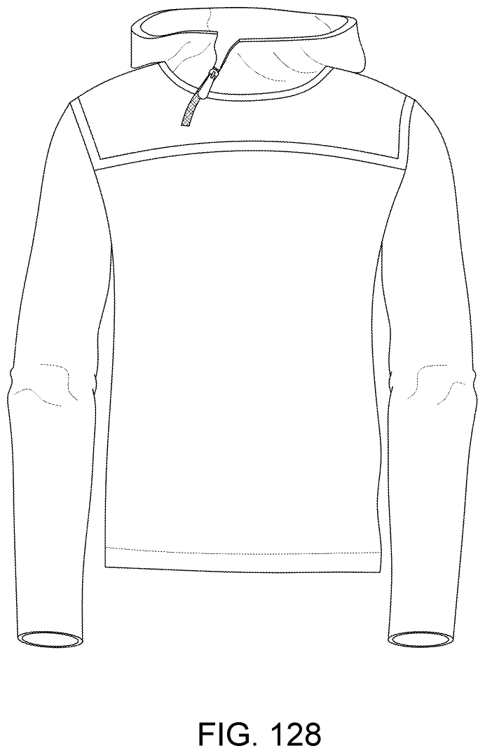

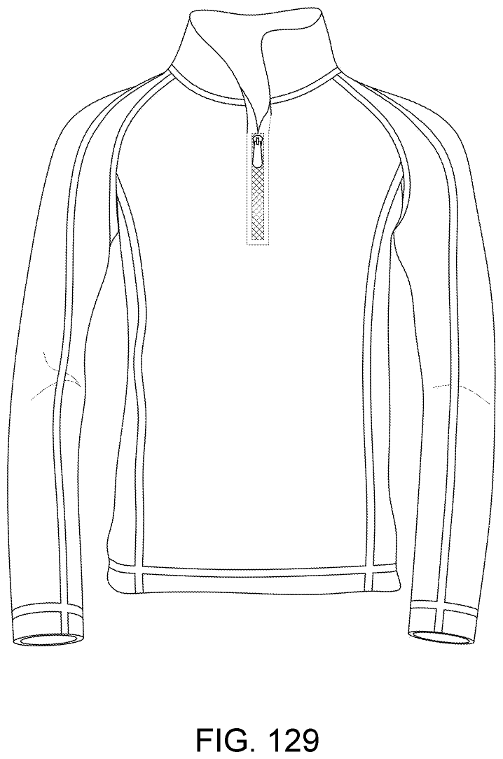

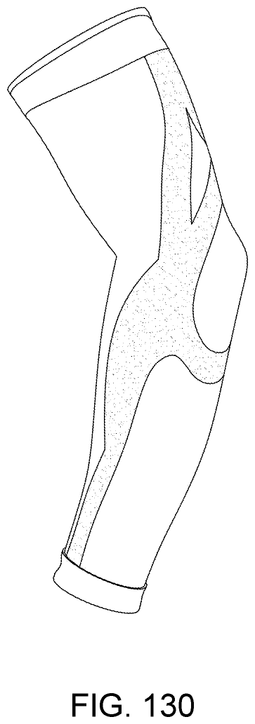

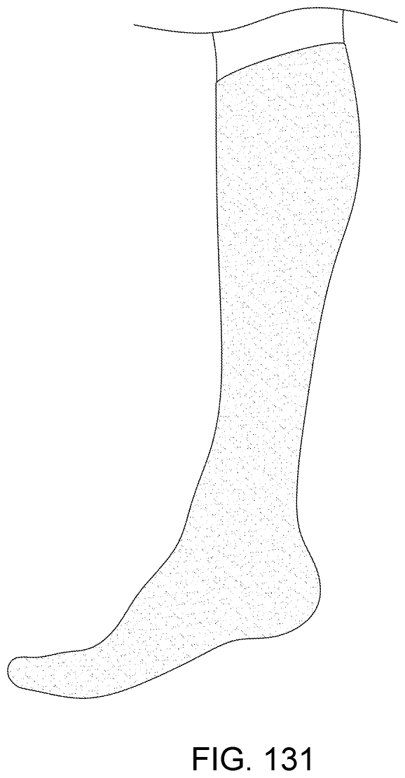

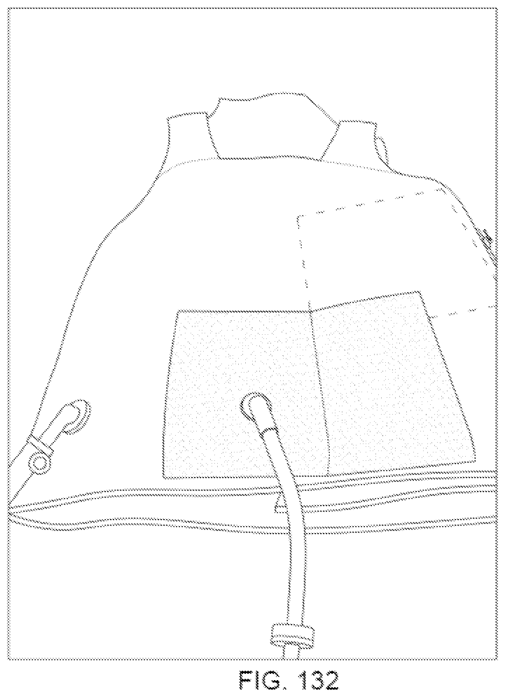

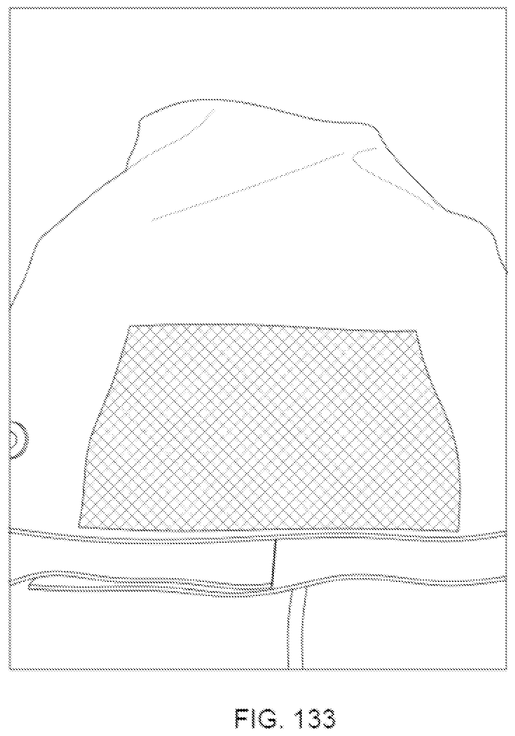

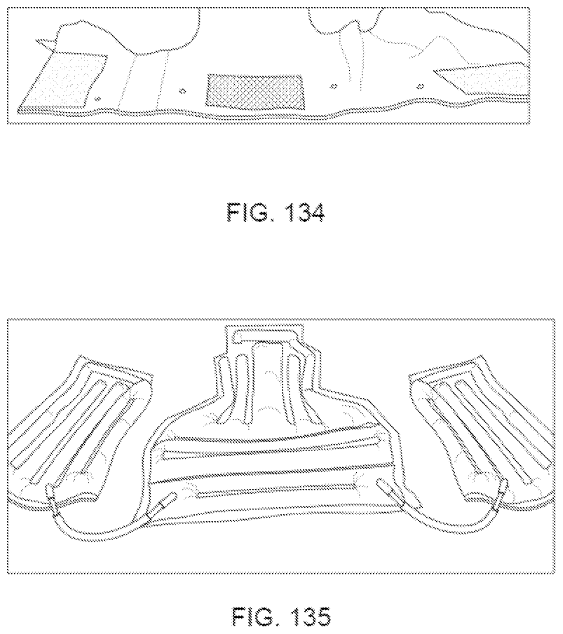

Referring to FIGS. 1-12, one aspect of the present invention is directed to garments, articles of clothing, and body equipment systems having a bladder system for providing better fit of the garments, articles of clothing, and body equipment systems to certain applicable body part(s) or area(s) of a user during use, and for providing greater support, comfort and/or stability to certain applicable body part(s) or area(s) of a user during use. As a non-limiting example, the present invention is directed to compression garments having a clothing component and a bladder system, compression articles of clothing having a clothing component and a bladder system, and also to compression body equipment systems having a bladder system for providing better fit of the compression garments, compression articles of clothing, and compression body equipment systems to certain applicable body part(s) or area(s) of a user during use, and for providing greater support, comfort and/or stability to certain applicable body part(s) or area(s) of a user during use.

As a non-limiting example or preferred embodiment of a compression garment or article of clothing 100, the clothing component 110, 130 may be a compression or non-compression top clothing component 110 (such as, but not limited to, a shirt (sleeveless, short sleeve, or long sleeve), a sweatshirt (sleeveless, short sleeve, or long sleeve), a jogging suit top, a sport's jersey, a sport's sweater, a vest, a jacket, a sweater, or a coat) and/or a compression or non-compression bottom clothing component 130 (such as, but not limited to, a casual short, an athletic or sport short (such as, but not limited to, a bicycling short, a lacrosse short, or a rugby short), a casual pant, an athletic or sport pant (such as, but not limited to, a bicycling pant, a baseball pant, or a football pant), a sweatpant, a jogging suit bottom, a legging, or a sock).

Preferably, the material of the clothing component 110, 130 can be made or manufactured in various designs, shapes, sizes, thicknesses, and weights.

As a non-limiting example, the material of the clothing component 110, 130 may be made or manufactured of lycra, polyester, spandex, cotton, wool, leather, silk, plastic, rubber, other natural fibers, other synthetic fibers, any material known to one of ordinary skill in the art (OOSIA), any compression material known to OOSIA, or any combination thereof.

Preferably, the compression material of the compression top clothing component 110 and compression bottom clothing component 130 of the compression garment or article of clothing 100 can be made or manufactured in various thicknesses and various levels of compression, and of any compression material known to OOSIA.

The bladder system 150 may be an air bladder system, a fluid bladder system, a combination air and fluid bladder system, any bladder system known to OOSIA, or any combination thereof.

Preferably and as a non-limiting example, the bladder system is an air bladder system 150 that comprises at least one bladder 160 wherein each bladder 160 has at least one chamber 170 dimensioned and configured for receiving air from an air source and adapted for being increased or decreased according to the user's preference and/or need of compression forces directed to certain applicable body part(s) or area(s) of a user or wearer during use to obtain better or greater fit, protection, support, comfort and/or stability to the applicable body part(s) or area(s) of the user. As a non-limiting example and as shown in FIGS. ???, the at least one bladder 160 has multiple, separated chambers 170 positioned at different locations along the bladder 160 so that different applicable body part(s) or area(s) of the user can possibly receive different amounts of compression forces directed to the applicable body part(s) or area(s) during use. As a non-limiting example, the user may wish to receive a higher amount of compression forces directed to the user's back as compared to the user's sides or front.

Alternatively and as a non-limiting example as shown in FIGS. 9, 10 and 12, the air bladder system 150 comprises multiple bladders 160 wherein each bladder 160 has at least one chamber 170. As non-limiting examples, each chamber 170 may be in communication with or in connection with at least one inlet 172 and/or at least one outlet 174, or multiple chambers 170 can be in communication with or in connection with at least one other chamber 170 and share at least one inlet 172 and/or at least one outlet 174. The air bladder system 150 may or may not include an air source 180, such as, but not limited to, an air pump, an air supply container, any air source known to OOSIA, or any combination thereof. As a non-limiting example, the air source 180 provides air to the corresponding chamber(s) 170 via at least one corresponding inlet 172, and, preferably, also via at least one corresponding connecting hose 176.

As a non-limiting example, the at least one bladder 160 may be interwoven within and/or between the fabric(s) or material(s) (or between layers of the fabric(s) or material(s)) of the clothing component 110, 130.

Alternatively, the at least one bladder 160 may be secured (by sewing, stitching, Velcro, hook and loop attachment, glue, or any securing method known to OOSIA) or positioned between two or more layers of the fabric or material of the clothing component 110, 130, or may be secured or positioned in front of or behind the fabric or material of the clothing component 110, 130.

Preferably, the at least one bladder 160 has a sufficient thickness or thinness to allow the compression garment or article of clothing 100 to provide better or greater fit, protection, support, comfort and/or stability to the applicable body part(s) or area(s) of the user during use while preferably also not being too bulky or heavy for the user. The at least one bladder 160 can be placed or positioned anywhere along the clothing component 110, 130, and the at least one chamber 170 can be positioned in various positions, designs and/or configurations depending on the sport, on the body areas that are desired to receive better or greater fit, protection, support, comfort and/or stability, and/or on the preference of the user.

As a non-limiting example, the at least one bladder 160 may be made or manufactured of a rubber material, a plastic material, any material known to OOSIA, and any combination thereof.

As a non-limiting example and as shown in FIGS. ???, the air bladder system 150 helps the compression garment or article of clothing 100 to provide better or greater fit (such as, but not limited to, any area that are loose or have extra spacing), protection, support, comfort and/or stability to certain applicable body part(s) or area(s) of a user or wearer during use by having a bladder 160 with multiple chambers 170 that can be designed in vertical, horizontal, any other configuration, and any combination configuration thereof).

The air bladder device system 150 includes hoses wherein the hoses and valves may be secured to the front, rear, sides, and/or any combination of positions of the bladder vest(s)/device(s).

Referring to FIGS. ???, another aspect of the present invention is directed to a compression body equipment system 200 having a body equipment component 210, 230 and a bladder system 250 for providing better or greater fit, protection, support, comfort and/or stability to certain applicable body part(s) or area(s) of a user or wearer during use.

As a non-limiting example or preferred embodiment of a compression body equipment system 200, the body equipment component 210, 230 may be a compression or non-compression top equipment component 210 (such as, but not limited to, any helmet (such as, but not limited to, a football helmet, a baseball/softball helmet, a skateboarding helmet, a skiing helmet, a motorcycle helmet, a bicycle helmet, or a construction worker helmet), a headgear, a neck protector, a throat protector, an arm sleeve, a football shoulder pad, or a baseball catcher chest protector) and/or a compression or non-compression bottom equipment component 230 (such as, but not limited to, a baseball catcher leg protector).

Preferably, the material of the body equipment component 210, 230 can be made or manufactured in various designs, shapes, sizes, thicknesses, and weights.

As a non-limiting example, the material of the body equipment component 210, 230 may be made or manufactured of leather, plastic, rubber, other natural fibers, other synthetic fibers, any material known to one of ordinary skill in the art (OOSIA), any compression material known to OOSIA, or any combination thereof.

Preferably, the compression material of the compression top equipment component 210 and compression bottom equipment component 230 of the body equipment system 200 can be made or manufactured in various thicknesses and various levels of compression, and of any compression material known to OOSIA.

The bladder system 250 may be an air bladder system, a fluid bladder system, a combination air and fluid bladder system, any bladder system known to OOSIA, or any combination thereof. Preferably, the bladder system 250 of the compression body equipment system 200 is substantially similar to or exactly the same as the bladder system 150 of the compression garment or article of clothing 100.

Preferably and as a non-limiting example, the bladder system is an air bladder system 250 that comprises at least one bladder 260 wherein each bladder 260 has at least one chamber 270 dimensioned and configured for receiving air from an air source and adapted for being increased or decreased according to the user's preference and/or need of compression forces directed to certain applicable body part(s) or area(s) of a user or wearer during use to obtain better or greater fit, protection, support, comfort and/or stability to the applicable body part(s) or area(s) of the user. As a non-limiting example and as shown in FIGS. ???, the at least one bladder 260 has multiple, separated chambers 270 positioned at different locations along the bladder 260 so that different applicable body part(s) or area(s) of the user can possibly receive different amounts of compression forces directed to the applicable body part(s) or area(s) during use. As a non-limiting example, the user may wish to receive a higher amount of compression forces directed to the user's front as compared to the user's sides or back.

Alternatively and as a non-limiting example, the air bladder system 250 comprises multiple bladders 260 wherein each bladder 260 has at least one chamber 270. As non-limiting examples, each chamber 270 may be in communication with or in connection with at least one inlet 272 and/or at least one outlet 274, or multiple chambers 270 can be in communication with or in connection with at least one other chamber 270 and share at least one inlet 272 and/or at least one outlet 274. The air bladder system 250 may or may not include an air source 280, such as, but not limited to, an air pump or similar device. As a non-limiting example, the air source 280 provides air to the corresponding chamber(s) 270 via at least one corresponding inlet 272, and, preferably, also via at least one corresponding connecting hose 276.

As a non-limiting example, the at least one bladder 260 may be interwoven within and/or between the fabric(s) or material(s) (or between layers of the fabric(s) or material(s)) of the body equipment system 200.

Alternatively, the at least one bladder 260 may be secured (by sewing, stitching, Velcro, hook and loop attachment, glue, or any securing method known to OOSIA) or positioned between two or more layers of the fabric or material of the body equipment system 200, or may be secured or positioned in front of or behind the fabric or material of the body equipment system 200.

Preferably, the at least one bladder 260 has a sufficient thickness or thinness to allow the body equipment system 200 to provide better or greater fit, protection, support, comfort and/or stability to the applicable body part(s) or area(s) of the user during use while preferably also not being too bulky or heavy for the user. The at least one bladder 260 can be placed or positioned anywhere along the body equipment system 200, and the at least one chamber 270 can be positioned in various designs and/or configurations depending on the sport, the body areas that are desired to be covered and/or protected, and/or the comfortability of the user as well.

As a non-limiting example, the at least one bladder 260 may be made or manufactured of a rubber material, a plastic material, any material known to OOSIA, and any combination thereof.

As a non-limiting example and as shown in FIGS. ???, the air bladder system 250 helps the body equipment system 200 to provide better or greater fit (such as, but not limited to, any area that are loose or have extra spacing), protection, support, comfort and/or stability to certain applicable body part(s) or area(s) of a user or wearer during use by having a compartmentalized bladder 260 (which has multiple chambers 270 that can be designed in vertical, horizontal, any other configuration, and any combination configuration thereof).

It is to be understood that the present invention is not limited to the embodiments described above or as shown in the attached figures, but encompasses any and all embodiments within the spirit of the invention.

1. A garment comprising:
a clothing component; and
a bladder system.

2. A body equipment system comprising:
a wearing component; and
a bladder system.

3. A compression garment comprising:
a compression clothing component; and
a bladder system.
